SEQUENCE LISTING (14 sequences)

SEQ ID NO: 1 - protein, 348 aa, Homo sapiens
  human constitutive androstane receptor (CAR) alpha (hCARa)
MASREDELRNCVVCGDQATGYHFNALTCEGCKGFFRRTVSKSIGPTCPFAGSCEVSKTQRRHCPACRLQKCLDAGMRKDMILSAEALALRRAKQAQRRAQQTPVQLSKEQEELIRTLLGAHTRHMGTMFEQFVQFRPPAHLFIHHQPLPTLAPVLPLVTHFADINTFMVLQVIKFTKDLPVFRSLPIEDQISLLKGAAVEICHIVLNTTFCLQTQNFLCGPLRYTIEDGARVGFQVEFLELLFHFHGTLRKLQLQEPEYVLLAAMALFSPDRPGVTQRDEIDQLQEEMALTLQSYIKGQQRRPRDRFLYAKLLGLLAELRSINEAYGYQIQHIQGLSAMMPLLQEICS

SEQ ID NO: 2 - protein, 358 aa, Mus musculus
  mouse constitutive androstane receptor (CAR) beta 1 (mCARbeta1, mCAR1)
MTAMLTLETMASEEEYGPRNCVVCGDRATGYHFHALTCEGCKGFFRRTVSKTIGPICPFAGRCEVSKAQRRHCPACRLQKCLNVGMRKDMILSAEALALRRARQAQRRAEKASLQLNQQQKELVQILLGAHTRHVGPLFDQFVQFKPPAYLFMHHRPFQPRGPVLPLLTHFADINTFMVQQIIKFTKDLPLFRSLTMEDQISLLKGAAVEILHISLNTTFCLQTENFFCGPLCYKMEDAVHAGFQYEFLESILHFHKNLKGLHLQEPEYVLMAATALFSPDRPGVTQREEIDQLQEEMALILNNHIMEQQSRLQSRFLYAKLMGLLADLRSINNAYSYELQRLEELSAMTPLLGEICS

SEQ ID NO: 3 - protein, 286 aa, Mus musculus
  mouse constitutive androstane receptor (CAR) beta 2 (mCARbeta2, mCAR2)
MTAMLTLETMASEEEYGPRNCVVCGDRATGYHFHALTCEGCKGFFRRTVSKTIGPICPFAGRCEVSKAQRRHCPACRLQKCLNVGMRKDMILSAEALALRRARQAQRRAEKASLQLNQQQKELVQILLGAHTRHVGPLFDQFVQFKPPAYLFMHHRPFQPRGPVLPLLTHFADINTFMVQQIIKFTKDLPLFRSLTMEDQISLLKGAAVEILHISLNTTFCLQTENFFCGPLCYKMEDAVHAGFQYEFLESILHFHKNLKGLHLQEPEYVLMAATALFSPGFCMQS

SEQ ID NO: 4 - DNA, 492 bp, Mus musculus
  murine CARbeta genomic sequence - Section A, portion of CARbeta intron sequence in left arm of targeting construct
aaaatttacccaacatagatttatctaatgtaattcctatctgcagaacatccaaatactttggaaattatttnttgtggttgtagctgtttgaatgtaaacatatattcaaaaaaactcttcatggtgatgtagcattgggcaagctatgaggatacctacttctggttatttactaaaagttgatagccaggcagtggtggcacacacctttaatcccagcacttgggaggcagaggcaggtggaattatgagtttgaggccagcctggtctacagagtgggttcaaggtcagccagggctacacagagaaaccctgtctcaaaaagaaggaggaggaggaggaaagaggaagaggaggaagaagatcttttgttttgagatagcatacagtgaaaatttcggtttctttagcaactcagttgtgtcacatgatgtctttctggaagctgtcttgtgagcagacatgtgatgtttatcacaatagaaagc

SEQ ID NO: 5 - DNA, 1779 bp, Mus musculus
  murine CARbeta genomic sequence - Section B, portion of CARbeta genomic sequence 5′ to Section A
aaagaggtcatcaggcttggcagcaagtgcctttgcctaccgagtctttacaccagctccaccgtggtttttgagacagtctcccactggactggatttcagcaagaaagctaggcttgccttcttgtctctgcctccttggcattggaattatgagttgttccaccgtgccatttttaaaaatgtaggttctaggaattaaactcggctctcggtgcttatatagtgagtactttacagagggagtcaccttgccagcacctagaattcacttttattcatatcccagtctccccacgtaagaaagtgggatcccttctagtgttacacctaagttcttagttggataccgaagtcttttttttaacagatctctggggctcagaaggcaagagctccttgcagaggatttaacctcaattcctagtactcaacttgccagctcataactgcctataactctagtcccagaagatcagacattgtcctctgatctctgtgggtactaggtatatacatttaaaaaaaatcaataaaaaatttaaaaaaagaaaagaaaaagaaagaaagaaaatcctttgggagcctggtataattgttatagctacctttttttttttttttttttttttttttaccatttgcaaactgcacgtgaaaaagcttgccatctctcccattgtttcctggcttattcaggatccatgcaaaaaggggagtgtagatttagcctaaagctcacccacagggaaatcctccaggagtctagtaagcagcagcttttaatgagtcatgaggtcctggcccctccccatctgccaccaaccaacacttctcgggcatgctaggaacccccaccccaccccacacccacacccaggtctttgccctgggtccagagtctgggtcctacctacatatggcaccgaggatacctagaggccccatgcaagagaaggcccttgttttccaggcactaaggaccgcagtccctaattcctggcagttcctgagatctcaaggaaagcagggtcagcgaggaggcctggggagaggaggcatcctacacccgatcttgtggcctgctgcctaagggaaacaggtaggtaatccgttggaggccagagacaaaaagcaacatttttgcttttaatgtcctcagtgctggggagcccggtgtcaggctgggcagtcttgggaagagattctgtagaggagagagaagagagtcctatggcccagtgctgattctcaactcctcccacattcaggagaccatgacagctatgctaacactagaaaccatggccagtgaagaagaatatgggccgaggaactgtgtggtgtgtggagaccgggccacaggctatcatttccacgccctgacttgtgagggctgcaagggcttcttcaggtgaatgcttcctccccaacagaaacaaccccgacatttctatcagtccacctttaaacactggtacacctccaagttataatcctcttgcagctaagctgcactgcccagtgtctagcactctcaatcttgctgaccacaacgcagtgtgaaactggtgacctaatgacaaggcaggttaaccatttgtcccagagacagagcctaagagtcaagaacacttgtgtagcacacactacctgcaaagcaccgagatgattgccacacgagggttcctgagtaaccttgtgttctcatgaaaacgctccaactacctctgaagacctttgagcacagctcagatgagtctgttgttaaatcgatcc

SEQ ID NO: 6 - DNA, 485 bp, Mus musculus
  murine CARbeta genomic sequence - Section C, CARbeta intron sequence in right arm of targeting construct
tgcattgctttctactgaagtgtatcacagatgaatatgagatcgacagaaagtgtgcagggatccccctgccatctggaaacacttaattcaatgaagtcccaaggaagcctcagaaactctttcttccttcctccttccttatctggggaggtggagtggccccaactgaagggatggctgaaaggtgctcgctgctgttctcaacagctttgtcatctctcttgcctgacacagtgatactgtcagcagaagccctggcattgcggcgagccagacaggcacagcggcgggcagagaaagcatctttgcaactgaatcagcagcagaaagaactggtccagatcctcctcggggcccacactcgccatgtgggccccatgtttgaccagtttgtgcagttcaaggtgagaacttaaccaggatgtgacctgggtacctgaggaggtaacccacagaagaaggctatgccctgatggaggaca

SEQ ID NO: 7 - protein, 8 aa, Artificial Sequence
  Description of Artificial Sequencesensor peptide, rhodamine-labeled peptide derived from the receptor binding domain of coactivator SRC-1
ILRKLLQE

SEQ ID NO: 8 - DNA, 1390 bp, Mesocricetus auratus
  hamster constitutive androstane receptor (CAR)
cttgttttctagggaccaaggacaatccctaattcctgcagttcctgagaccacaaggaaagcagggtcatcgtggaggcttggagacaggcatctcataccagattttgtgacctgcgtgtgtcatactgcctaagagaaacaggagaccatgacagctacgctaacactcgaaaccaaggccagtggagaggaatatggaccgaggaactgtgtggtgtgtggagaccgagccacgggctaccatttccatgccctgacttgtgagggctgcaaaggcttcttcagacgaactgtcagcaaaaccattagtcccatctgtccattttctggaagctgtgagatcagcagagcccagagacgccactgcccagcctgcaggttgcagaagtgcctaaacgctggcatgaggaaagacatgatactgtcagcagaagccctgtcgttgcggcgagccaggcaggcacagcggcgggcacaaaaagcttccgtgcagatgactcaggagcggaaggagctggtccagaccctcataggggcccacacccgccacatgggccccatgtttgaccagtttgtgaagctcaggcctccagcttacctgttcacccatcaccggccctcctccccgctggtcccccccgcgttaccactgctcacacactttgcagatgtcaacactttcatggtgcagcagattatcaagttcaccaaggaactgcccctttttcggtccctacccgtggaggaccagatctcccttctcaagggagcagctgtggaaatattgcatatctcactcaacactactttctgtcttcaaacacagaatttcttctgtgggccactttgctacaaaatggaagacgcagcccacgcagggttccggtacgaatatgtggagttgatctttcgcttccatgggacactgaagcgactgcagctccaagagcctgagtatgtgctcatgactgccatggccctcttctctcctgacaggcctggaatcacccagagagaagagattgaccagctgcaagaggagatggcactgattttgaacaactacattatggaacagcagccaaggccccagagtcggtttctgtacgcaaagctgatgggcctgctggctgagctccggagcataaacaatgcatactcatatgaaatacggcgcatccagggactgtccgctatgatgccactacttggggaaatctgcagctgaggctcaggcttgcctccttccccagggcccctgggattcattggactggaaaggggaaattgctgagctaaaaggagctcagtgacagcaaaaaacactggacagtnggaaaaaaannnnnnnnnnnnnaaaagcgacctgcccgggcggccgttcagc

SEQ ID NO: 9 - protein, 359 aa, Mesocricetus auratus
  predicted amino acid sequence of hamster constitutive androstane receptor (CAR)
MTATLTLETKASGEEYGPRNCVVCGDRATGYHFHALTCEGCKGFFRRTVSKTISPICPFSGSCEISRAQRRHCPACRLQKCLNAGMRKDMILSAEALSLRRARQAQRRAQKASVQMTQERKELVQTLIGAHTRHMGPMFDQFVKLRPPAYLFTHHRPSSPLVPPALPLLTHFADVNTFMVQQIIKFTKELPLFRSLPVEDQISLLKGAAVEILHISLNTTFCLQTQNFFCGPLCYKMEDAAHAGFRYEYVELIFRFHGTLKRLQLQEPEYVLMTAMALFSPDRPGITQREEIDQLQEEMALILNNYIMEQQPRPQSRFLYAKLMGLLAELRSINNAYSYEIRRIQGLSAMMPLLGEICS

SEQ ID NO: 10 - DNA, 58 bp, Artificial Sequence
  Description of Artificial Sequenceoverlapping Oligo 2930
ccataaacgtgttgatatctgcaaagtgtgcgagcagaggcaacacggggccccgagg

SEQ ID NO: 11 - DNA, 58 bp, Artificial Sequence
  Description of Artificial Sequenceoverlapping Oligo 2931
ctctacagcctccagcctatctgttcatgcatcaccggcctttccagcctcggggccc

SEQ ID NO: 12 - DNA, 21 bp, Artificial Sequence
  Description of Artificial Sequenceboundary of the deleted region of the mouse CARbeta gene in CAR knock-out construct
cactagaaaccatggccagtg

SEQ ID NO: 13 - DNA, 22 bp, Artificial Sequence
  Description of Artificial Sequenceboundary of the deleted region of the mouse CARbeta gene in CAR knock-out construct
tgtgtggtgtgggagaccgggc

SEQ ID NO: 14 - protein, 201 aa, Artificial Sequence
  Description of Artificial Sequenceflexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGPGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

(19) United States
(12) Patent Application Publication
(10) Pub. No.: US 2002/0152479 A1
(43) Pub. Date: Oct. 17, 2002

(54) Car modulators: screening and treatment of hypercholesterolemia

(76) Inventors: Jurgen M. Lehmann, San Carlos, CA (US); Andrew Kwan-Nan Shiau, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/760,364

(22) Filed: Jan. 12, 2001

Related U.S. Application Data
(60) Provisional application No. 60/176,398, filed on Jan. 13, 2000.

(51) Int. Cl. A61K 31/57; G01N 33/00; A01K 67/00

(52) U.S. Cl. 800/3; 514/177

(57) ABSTRACT

This invention provides methods that are useful for identifying therapeutic agents for the treatment of a CAR-mediated disorder or condition. The methods include determining whether the candidate therapeutic agent can: interact directly with CAR, modulate CAR-mediated gene expression, decrease CAR antagonist elevation of a cholesterol indicator in a mammal, or decrease a cholesterol level indicator in a mammal with a defective CAR. Also provided are CAR agonists.

### CROSS-REFERENCES TO RELATED APPLICATIONS

This application claims the benefit of U.S. Provisional Patent application Ser. No. 60/176,398, filed on Jan. 13, 2000, the teachings of which are herein incorporated by reference.

### BACKGROUND OF THE INVENTION

1. Field of the Invention

This invention pertains to the field of identifying therapeutic agents for the treatment of a disorder that is mediated by the constitutive androstane receptor (CAR), such as hypercholesterolemia. Provided are methods for carrying out screening assays to identify candidate therapeutic agents for treating CAR-mediated disorders, as well as methods of treating CAR-mediated disorders.

2. Background

Atherosclerosis is a leading cause of death, myocardial infarctions, stroke, peripheral vascular disease and cardiovascular disease (Libby, in Chapter 242 of Harrison's Principles of Internal Medicine, 14th edition (1998) (Fauci et al., eds.); Witztum, in Chapter 36 of Goodman & Gilman 's The Pharmacological Basis of Therapeutics, 9th edition (1996) (Hardman et al., eds.)). One of the major contributing factors to atherosclerosis is hypercholesterolemia.

Hypercholesterolemia is currently treated with a combination of dietary and pharmaceutical therapies. However, these treatments have significant disadvantages. Often, more than a single pharmaceutical agent and a dietary regimen are necessary to decrease total cholesterol and LDL cholesterol levels to the desired level. Drugs such as bile acid sequestrants, niacin and the statins are commonly used to treat hypercholesterolemia and atherosclerosis. The use of niacin, for example, is limited by the high incidence (>50%) of numerous side effects that are experienced in patients. Thus, a need for therapeutic agents that would decrease cholesterol levels still exists.

The development of improved treatments for hypercholesteremia has been hampered by the lack of understanding as to the pathways that synthesize and degrade cholesterol. Most of the current target enzyme and proteins have been discovered using biochemical and genetic techniques. The statins are competitive inhibitors of 3-hydroxy-3-methylglutaryl coenzyme A (HMG CoA) reductase, which catalyzes the rate limiting step in cholesterol biosynthesis, and have proved to be very useful in treating hypercholesterolemia. Also, it is known that mutations in the LDL receptor gene can give rise to familial hypercholesterolemia. But the discovery of additional steps that regulate cholesterol levels is desirable as not all of the causes of hypercholesterolemia or the mechanisms involved have been elucidated.

Potential targets for therapeutic agents that modulate cholesterol levels include the hormones that modulate eukaryotic cell processes and the molecules that bind them. Development and homeostasis in complex eukaryotes, including humans and other mammals, birds, and fish, are regulated by a wide variety of substances, including hormones or ligands produced by a cell, or compounds taken up from regular diet (e.g., bile acids, oxysterols, and fatty acids). The effects of these hormones are mediated through binding of the hormone to a specific, high affinity receptor or nuclear receptor.

Nuclear receptors form a large family of ligand-activated transcription factors that modify the expression of target genes by binding to specific cis-acting sequences (Laudet et al. (1992) EMBO J. 11: 1003-1013; Lopes da Silva et al. (1995) TINS 18: 542-548; Mangelsdorf et al. (1995) Cell 83: 835-839; Mangelsdorf et al. (1995) Cell 83: 841-850). These receptors, which include the thyroid hormone, retinoid, fatty acid, and eicosanoid receptors, typically bind to their cognate response elements as heterodimers with a 9-cis-retinoic acid receptor (RXR). Often, binding of a nuclear receptor to a response element occurs in the absence of the cognate ligand.

One such nuclear receptor is the constitutive androstane receptor (CAR, which is also known as the constitutively active receptor. CARα was originally cloned from a human liver cDNA library and was initially termed MB67 (Baes et al. (1994) Mol. Cell. Biol. 14: 1544-1552; U.S. Pat. No. 5,756,448; GenBank accession number Z30425). Initially, no ligand was known for the CAR/MB67 protein, so it was classified as an orphan receptor. Based on its amino acid sequence, CARα was classified as a member of the nuclear receptor superfamily. Unlike other known members of the nuclear receptor family, CARα is able to activate transcription in the absence of exogenously added ligand (Baes et al., supra).

CARα is able to activate transcription from a subset of retinoic acid response elements (RAREs) such as the RAREs from the human retinoic acid receptor beta (RARE) and the human alcohol dehydrogenase 3 (ADH3) genes without any exogenous ligand (Baes et al., supra). These response elements are direct repeats of hexamers related to the 5′AGGTCA3′ sequence that are separated by 5 base pairs (the DR-5 class of elements). CARα appeared to be a new heterodimer partner for the retinoid X receptor (RXR) that was able to activate a subset of RAREs in a retinoid independent manner (Baes et al., supra).

CAR response elements have also been found in the promoters of the genes encoding the cytochrome P-450 CYP2B (Sueyoshi et al. (1999) J. Biol. Chem. 274: 6043-6046). Phenobarbitol as well as a large group of structurally and functionally diverse xenobiotics are known to induce expression of CYP2B genes. Phenobarbitol causes nuclear translocation of CAR (Kawamoto et al. (1999) Mol. Cell. Biol. 19: 6318-6322). The CAR response elements were described as belonging to the DR-4 class (i.e., direct repeats of hexamers separated by 4 base pairs). CAR reportedly activates transcription of the cytochrome P-450 gene CY2B by binding to cognate DNA sequences in the CYP2B promoter region as a heterodimer with RXR (Honkakoski et al. (1998) Mol. Cell. Biol. 18: 5652-5658). Androstenol decreases CAR-mediated induction of the CYB2B gene (Sueyoshi et al., supra).

The murine homolog of CARα, which is termed CARβ, was cloned from a mouse liver cDNA library (Choi et al. (1997) J. Biol. Chem. 272: 23565-23571; GenBank Accession No. AF009327). CARα shares 72% and 88% amino acid homology with CARβ in the ligand binding domain and DNA binding domains, respectively. Later, it was found that androstane metabolites have the ability to bind to CARα and CARβ (Forman et al. (1998) Nature 395:612-615; WO 96/36320). The constitutive transcriptional activity of CARβ and CARα is inhibited by the compounds 5α-androst-16-en-3α-ol and 5α-androstan-3α-ol. Thus, these compounds act as inverse agonists of CAR (see Forman et al., supra; WO 96/36230, supra). The compounds androstenol-3-acetate and 5α-androstan-3α-ol-acetate are also able to inhibit CAR's constitutive transcriptional activity. Androstenol and androstanol do not appear to inhibit the DNA binding properties of CAR, but can cause the dissociation of the co-activator SRC-1 from CAR. CAR is also active as a transcriptional activator in yeast, which do not have endogenous steroid signaling pathways. Androstanol was also able to inhibit CAR activity in a yeast assay (Forman et al., supra). To date, however, CAR has not been linked to any disease or condition.

The discovery and design of cholesterol lowering agents has been hampered because the physiological mechanisms that regulate cholesterol levels are not completely understood. Thus, a need exists for the identification of hormones and other molecules that regulate cholesterol levels, as well as for methods for identifying agents that can modulate cholesterol levels. The present invention fulfills these and other needs.

### SUMMARY OF THE INVENTION

The present invention provides methods for identifying therapeutic agents for use in treating a CAR-mediated disorder or condition. CAR-mediated disorders or condition include, for example, hypercholesterolemia, lipid disorders, atherosclerosis, and cardiovascular disorders. The methods involve determining whether the candidate therapeutic agent modulates CAR-mediated regulation of cholesterol levels. In some embodiments, the method further involves pre-screening candidate therapeutic agents by assessing the ability of the candidate therapeutic agents to modulate intermolecular interactions between CAR and a ligand for CAR. For example, the screening can involve determining whether a compound can modulate CAR-mediated gene expression, or can increase or decrease the ability of CAR to bind to a ligand for CAR.

Also provided by the present invention are methods for treating a CAR-mediated disorder or condition. These methods involve administering to a mammal having a CAR-mediated disorder or condition a therapeutically effective amount of an agent that modulates CAR-mediated regulation of cholesterol levels. In a presently preferred embodiment, the CAR-mediated disorder or condition is hypercholesterolemia, lipid disorders, atherosclerosis, or cardiovascular disorders.

In another aspect, the present invention provides a non-human mammal having a genome with a disruption in at least one CAR allele.

Also provided by the present invention, is a non-human mammal having a genome with a disruption in both CAR alleles. In a presently preferred embodiment, the non-human mammal having a genome with a disruption in both CAR alleles exhibits an increased level of serum cholesterol relative to a wild-type mammal.

The present invention also provides methods for producing a transgenic non-human mammal having a genome with a disrupted CAR allele. These methods typically involve introducing into embryonic stem cells a polynucleotide that contains a coding region for a portion of a CAR polypeptide. The polynucleotide sequence includes a disruption in the coding region of a CAR gene and insertion of a selection marker, such as the neomycin resistance gene. A cell is then identified into which the polynucleotide sequence has been integrated into an endogenous CAR allele. The cell is then introduced into a blastocyst. The transgenic blastocyst is implanted into a pseudopregnant mammal, which is allowed to give birth to a transgenic mammal.

### BRIEF DESCRIPTION OF THE DRAWINGS

FIG. 1 is a schematic of an approach for the disruption of the mouse CARβ gene. The mouse cDNA is shown schematically, with the ATG denoting the start codon. The small bar immediately below the mouse cDNA represents the nucleic acid fragment that was used to probe Northern blots to ascertain whether the CAR allele has been disrupted. The DNA binding domain of CAR is indicated as forward hatched areas. The genomic structure of the CAR knockout construct is depicted below the cDNA schematic, shown with the Neo^{R} gene inserted as used to construct the knockout mouse. The Neo^{R} gene is transcribed in the direction of the arrow and is depicted as a reverse hatched area. The numbers refer to nucleotide positions in the CARβ cDNA. The lower case nucleotides indicate the boundaries of the deleted region of the CARβ gene.

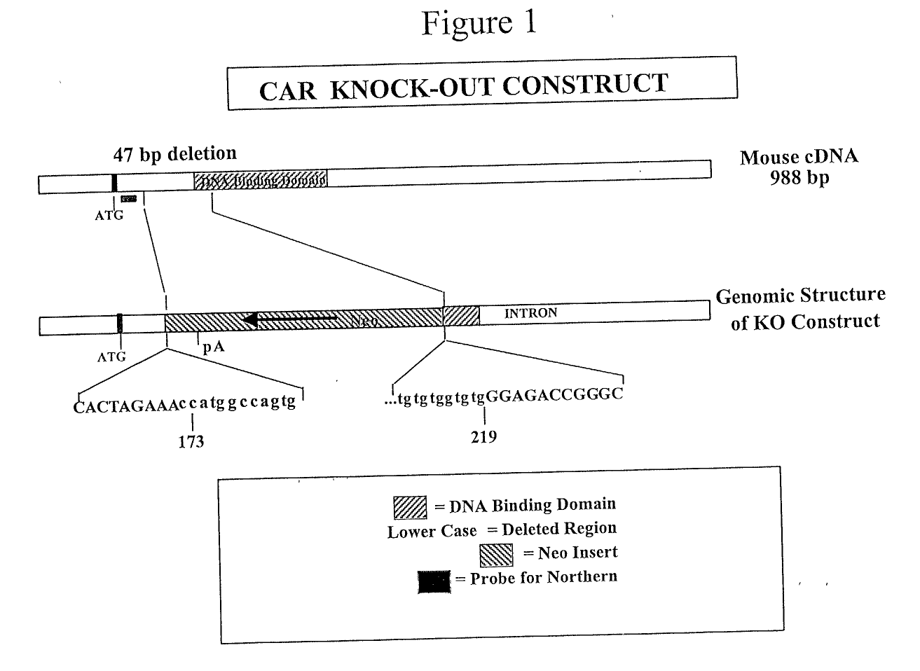

FIG. 2 shows a schematic of the structure of the genomic DNA sequence of CARβ, which includes intron sequences, a DNA binding domain encoding sequence and the sequence deleted by Neo^{R} insertion in pc155. The lower case letters represent intron sequences. Section A (SEQ ID NO: 4) is a portion of CARβ intron sequence in the left arm of the targeting construct. Section B (SEQ ID NO: 5) depicts a portion of the CARβ genomic sequence that is 5′ of Section A. The upper case letters denote the protein coding region of the CARβ gene and the DNA binding domain is underlined. The bolded and italicized upper case letters denote the CARβ nucleotide sequences deleted and replaced by the Neo^{R} sequence in pc155. The deletion starts 22 bp 3′ of the initiator ATG of CARβ and deletes 47 bp of CARβ sequence. The Neo^{R} insertion deletes nucleotides 173 to 219 of murine CAR, which corresponds to amino acids 21-86 (GenBank Accession No. AF009327). Section C (SEQ ID NO: 6) is CARβ intron sequence contained in the right arm of the targeting construct.

FIG. 3 is shows the ability of 5β-pregnan-3,20-dione to increase the fluorescence polarization between rhodamine labeled ILRKLLQE, rhodamine-ILRKLLQE, and a GST-CARα ligand binding domain fusion protein. The concentration of 5β-pregnan-3,20-dione is on the x-axis.

FIG. 4 demonstrates that the androstane compounds 5α-androst-16-en-3α-ol and 5α-androstan-3α-ol are able to decrease the amount of fluorescence polarization seen with 5β-pregnan-3,20-dione, rhodamine-ILRKLLQE, and a GST-CARα ligand binding domain fusion protein (GST-CARα). The assay was performed with 5α-androst-16-en-3α-ol (filled squares) or 5α-androstan-3α-ol (filled circles) incubated in the presence of rhodamine-ILRKLLQE, and GST-CARα. The IC₅₀ is the concentration at which 50% of the fluorescence polarization seen in the absence of the androstanes is inhibited. The assay was also performed with 5α-androst-16-en-3α-ol (open squares) or 5α-androstan-3α-ol (open circles) incubated in the presence of 5 μM 5β-pregnan-3,20-dione, rhodamine-ILRKLLQE, and GST-CARα. The IC₅₀ is the concentration at which 50% of the fluorescence polarization seen with 5 μM 5β-pregnan-3,20-dione in the absence of the androstans is inhibited.

FIG. 5 demonstrates the activation of CAR dependent transcription in a reporter gene assay by 5β-pregnan-3,20-dione. Reporter plasmid, (pRAREβ-tk luc) a β-galactosidase expression vector (pCMVβ, Clontech), and increasing amounts of pCMV-CARα mammalian expression vector were transfected into CV-1 cells. The transfected cells were incubated with vehicle (open bars), 10 μM 5α-androstan-3α-ol (black bars), 10 μM 5β-pregnan-3,20-dione (gray bars) or 10 μM 5α-androstan-3α-ol and 10 μM 5β-pregnan-3,20-dione (hatched bars). The cells were assayed for luciferase and β-galactosidase activity. The luciferase activity is reported as normalized luciferase activity which is the ratio of luciferase activity to β-galactosidase activity.

FIG. 6 shows that mice homozygous for a CAR allele disruption exhibit reduced tolerance to a dietary lipid challenge. Mice were fed a normal diet (PicoLab® 5053 containing 4% fat) or a high fat diet (PicoLab® 5053 containing 1.25% cholesterol and 10% coconut oil). Blood was withdrawn from the mice at 1, 8, 15 and 21 days and the serum was assayed for total cholesterol.

FIG. 7 demonstrates that TCPOBOP decreases plasma cholesterol concentrations in CAR wild-type mice (+/+) and mice heterozygous for a disrupted CAR allele (+/−), but not in CAR knockout mice (−/−). Male or female littermates received a single 0.2 ml intraperitoneal injection of 0.3 mg/ml TCPOBOP in corn oil or vehicle (corn oil) at a dose of 3 mg/kg. Plasma samples were analyzed for total plasma cholesterol. The right hand panel are female mice, the left hand panel are male mice. The open bars are mice treated with vehicle. The filled bars represent values from mice treated with TCPOBOP. The genotypes of the mice are depicted on the x-axis as +/+ (homozygous for both native CAR alleles), +/− (heterozygous—having one native CAR allele and one disrupted CAR allele), −/− (having disruptions in both CAR alleles).

FIG. 8 shows that TCPOBOP treatment reduces plasma levels of LDL/VLDL in wild-type CAR littermates, but not in mice that contain disruptions in both CAR alleles. Mice were injected with TCPOBOP in corn oil or vehicle (corn oil) as described in FIG. 7. HDL and LDL/VLDL levels were assayed by electrophoresing plasma samples using the Paragon® Lipoprotein Electrophoresis Kit (Beckmann Instruments, Inc., Fullerton, Calif.) and stained according to the manufacturer's instructions. The upper panel of FIG. 8 is the vehicle treated panel. The lower panel of FIG. 8 is the TCPOBOP treated panel. The sex is indicated by M (male) or F (female). The genotype is indicated by WT (wild-type for both CAR alleles) or KO (mice that contain a disruption in both CAR alleles). The migration positions of HDL and LDL/VLDL are indicated on the right hand side of the photograph of the gel.

FIG. 9 demonstrates that TCPOBOP decreases plasma cholesterol concentrations in wild-type C57B1/6 mice. Male or female C57B1/6 littermates received a single 0.2 ml intraperitoneal injection of 0.3 mg/ml TCPOBOP in corn oil or vehicle (corn oil). The open bars are mice treated with vehicle. The black bars are mice treated with TCPOBOP. The hatched bars are females treated with vehicle. The gray bars are females treated with TCPOBOP.

### DESCRIPTION OF THE SPECIFIC EMBODIMENTS

Definitions

The term “CAR” refers to a member of the nuclear hormone superfamily known as the constitutive androstane receptor, the constitutively active receptor, CARα, CARβ, MB67, or the hamster CAR (SEQ ID NO: 8). Generally, a CAR will have encompasses amino acid sequences that are at least 75% identical to the predicted amino acid sequence of human CARα (GenBank Accession No. Z30425), the predicted amino acid sequence of mouse CARβ (GenBank Accession number AAC53349) or the predicted amino acid sequence of the hamster CAR gene (SEQ ID NO: 9).

A “CAR polypeptide” is a polypeptide comprising an amino acid sequence that is substantially identical with a portion of a CAR amino acid sequence.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual alignment and inspection.

The phrase “substantially identical,” in the context of two nucleic acids or polypeptides, refers to two or more sequences or subsequences that have at least 75%, preferably 85%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions and/or untranslated regions.

For sequence comparison, typically one sequence acts as a reference sequence, to which test sequences are compared. When using a computerized sequence comparison algorithm, test and reference sequences are input into a computer, subsequence coordinates are designated, if necessary, and sequence algorithm program parameters are designated if changes from the default parameters are desired. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/).

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. (As the target sequences are generally present in excess, at Tₘ, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W. H. Freeman and Company.

The phrase “specifically” or “selectively” binds, when referring to a ligand/receptor or other binding pair, refers to a binding reaction which is determinative of the presence of the protein in the presence of a heterogeneous population of proteins and other biologics. Thus, under designated conditions, a specified ligand binds to a particular receptor and does not bind in a significant amount to other proteins present in the sample. Specific binding to a particular target receptor or antigen under such conditions may require a ligand or antibody that is selected for its specificity for the particular receptor. For example, antibodies raised to CAR polypeptides (or subsequences thereof) or to the polypeptides partially encoded by CAR polynucleotide sequences can be selected to obtain antibodies specifically immunoreactive with the full length proteins and not with other proteins except perhaps to polymorphic variants. A variety of immunoassay formats can be used to select antibodies specifically immunoreactive with a particular protein. For example, solid-phase ELISA immunoassays are routinely used to select monoclonal antibodies that are specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity. Typically a specific or selective reaction will provide a signal that is at least twice background signal or noise, and more typically more than 10 to 100 times background. Specific binding between members of a binding pair is indicated by an affinity of at least 50 μM. Preferred binding agents bind with an affinity of at least about 1 μM, more preferably of at least about 100 nM, and still more preferably of at least about 10 nM, and even more preferably of at least about 1 nM.

The phrase “CAR-mediated disorder or condition” refers to a pathology or disease caused by a CAR. One example of a CAR-mediated disorder or condition is hypercholesterolemia.

The phrase “CAR-mediated intermolecular interaction” refers to an interaction between CAR and another molecule. Such interactions include, without limitation, gene transcription and the binding of a molecule. CAR-mediated intermolecular interactions can be assayed by measuring one or more parameters that are indirectly or directly under the influence of CAR, e.g., functional, physical, physiological, and chemical effects. Such intermolecular interactions can be measured in an in vivo or in vitro assay by any means known to those skilled in the art, e.g., changes in spectroscopic characteristics (e.g., fluorescence, absorbance, refractive index) of CAR or any CAR associated molecules, hydrodynamic (e.g., shape), chromatographic, or solubility properties, changes in gene expression of CAR, or of any marker genes indicative of CAR activity, or any other detectable effect on a cell or an animal.

The term “CAR ligand” refers to a compound that binds specifically to a CAR polypeptide with an affinity constant (K_{d}) of around 20 μM or less more preferably about 1 μM or less, still more preferably about 100 nM or less, and most preferably 2 nM or less.

An “agonist” is a compound that interacts with a target and can cause an increase in the activation of the target. A compound that interferes with a CAR inverse agonist's effects on CAR, promotes CAR-mediated transcription, decreases cholesterol levels, or affects a cholesterol indicator is referred to as a “CAR agonist.”

An “inverse agonist” is a compound that acts on the same target as that of an agonist, but produces an opposite effect of the agonist. Inverse agonists are also termed negative agonists. Some compounds that are inverse agonists also act as antagonists in under certain conditions. For example, the CAR antagonists 5α-androst-16-en-3α-ol and 5α-androstane-3α-ol are antagonists of the effects of the CAR agonist 5β-pregnan-3,20-dione.

An “antagonist” is a compound that opposes the actions of an agonist. A “CAR antagonist” is a compound with the ability to inhibit CAR-mediated transcription, or to interfere with a CAR agonist's effect on CAR, increases cholesterol levels, or affects a cholesterol indicator.

The term “cholesterol levels” refers to the level of serum cholesterol in a subject or the level of cholesterol forms such as HDL cholesterol, LDL, cholesterol, and VLDL cholesterol, etc.

A “cholesterol-elevated mammal” is a mammal with a cholesterol level in the 95th percentile of the population. In humans, however, the level of cholesterol that would be described as “cholesterol-elevated” is a total cholesterol level in excess of about 200 mg/dl or in excess of about 130 mg/dl of LDL cholesterol.

The term “mammal” refers to a member of the class Mammalia. Examples of mammals include, without limitation, humans, primates, chimpanzees, rodents, mice, rats, rabbits, sheep, and cows.

A “cholesterol indicator” is a marker of the level of cholesterol present in a cell or a mammal. “Cholesterol indicator(s)” include, without limitation, the levels of serum cholesterol, HDL cholesterol, LDL cholesterol, VLDL cholesterol, and the level of a mRNA involved in the regulation of cholesterol levels.

A “CAR compromised mammal” is a mammal that has a diminished capacity to produce a functional CAR polypeptide or produces a CAR polypeptide with a diminished ability to act under a defined set of conditions. An example of a “CAR compromised mammal” is a mammal with a mutation, disruption, or insertion, in both CAR alleles that prevents the production of a functional CAR polypeptide.

A “CAR non-compromised mammal” is a mammal that produces enough functional CAR polypeptide such that the mammal does not suffer from a CAR-mediated disorder or condition.

The “DNA binding domain of CAR” is that portion of a CAR polypeptide that confers the ability of the CAR polypeptide to bind to a CAR responsive element, such as a DR-S element. The “DNA binding domain of CAR” is from around amino acid 11 to about amino acid 76 of CARα (GenBank Accession No. Z30425) and from around amino acid 21 to about amino acid 86 of CARβ (GenBank Accession No. AF009327).

The phrase “CAR-mediated gene expression” refers to transcription of a polynucleotide that is controlled, regulated, or modulated by a CAR polypeptide.

A “DNA binding domain of a polypeptide” is a polypeptide or region of a polypeptide that is able to bind to a DNA sequence. DNA binding domains can be found in polypeptides such as: a CAR, a GAL4, an estrogen receptor, a progesterone receptor, a glucocorticoid receptor, an androgen receptor, a mineralcorticoid receptor, a vitamin D receptor, a retinoid receptor, and a thyroid hormone receptor.

A “ligand binding domain” is a polypeptide or region of a polypeptide that is able to bind a compound. Example such compounds can be proteins, peptides, naturally occurring molecules, synthetic molecules, and combinations thereof.

A “reporter gene construct” is a polynucleotide is comprised of a response element, a promoter and a reporter gene. The “reporter gene” typically encodes for a protein or enzyme that can be detected. Examples of reporter genes include, without limitation, luciferase, alkaline phosphatase, beta-galactosidase, chloramphenicol acetyl-transferase, and the green fluorescent protein (GFP).

The phrase “operably linked” refers to a functional linkage between a nucleic acid expression control sequence (such as a promoter, or array of transcription factor binding sites) and a second nucleic acid sequence, wherein the expression control sequence directs the transcription of the nucleic acid corresponding to the second sequence.

A “response element” is a polynucleotide sequence that has the ability to bind a transcription factor.

A “CAR response element” is a polynucleotide sequence that has the ability to bind to a CAR polypeptide.

The term “DR-5” refers to an element comprised of a polynucleotide sequence having direct repeats of two half sites separated by 5 nucleotides. Each half site has about 80% or greater homology to the sequence 5′AGGTCA3′.

The term “DR-4” refers to an element comprised of a polynucleotide sequence having direct repeats of two half site hexamers separated by 4 nucleotides. Each half site has comprises the sequence 5′T(G/C)(T/A/G)(A/C)(A/C)(T/C)3′. Examples of DR-4 sequences can be found in the CYP2B gene.

The “binding of a candidate therapeutic agent directly to a CAR polypeptide” occurs when the candidate therapeutic agent has an affinity for a CAR polypeptide or a protein comprising a CAR ligand binding domain.

A “candidate therapeutic agent can inhibit the interaction between a CAR polypeptide and a CAR ligand” when the presence of candidate therapeutic agent decreases the amount of CAR ligand that can bind to a CAR polypeptide or a protein comprising a CAR ligand binding domain in a binding assay.

A composition is “labeled” that is detectable, either directly or indirectly, by spectroscopic, photochemical, biochemical, immunochemical, or chemical means. For example, useful labels include ³²P, fluorescent dyes, electron-dense reagents, enzymes and their substrates (e.g., as commonly used in enzyme-linked immunoassays, e.g., alkaline phosphatase and horse radish peroxidase), biotin-streptavidin, digoxigenin, or haptens and proteins for which antisera or monoclonal antibodies are available. The label or detectable moiety is typically bound, either covalently, through a linker or chemical bound, or through ionic, van der Waals or hydrogen bonds to the molecule to be detected.

The term “radiolabeled” refers to a compound to which a radioisotope has been attached through covalent or non-covalent means. Examples of radioisotopes include, without limitation, H³, P³³, P³², S³⁵, and I¹²⁵.

A “fluorophore” is a compound or moiety that accepts radiant energy of one wavelength and emits radiant energy of a second wavelength.

A “non-human mammal” includes members of the class Mammalia, except humans.

A “disruption in at least one CAR allele” is an aberration in at least one CAR allele as compared to a wild-type allele. A disruption can include, without limitation, an insertion, deletion or mutation.

### DESCRIPTION OF THE PREFERRED EMBODIMENTS

I. Introduction

The present invention provides compounds and methods that are useful for treating hypercholesterolemia and other disorders that involve aberrant cholesterol levels. The invention is based on the discovery that CAR (constitutive androstane receptor) is involved in modulation of serum cholesterol levels. Accordingly, the invention provides methods for identifying therapeutic agents that are useful in treating CAR-mediated conditions and disorders, as well as transgenic animals that exhibit such conditions and thus are useful for testing potential treatments and for studying the mechanisms of cholesterol regulation.

II. Methods for Identifying Compounds for Use in Treating Car-mediated Disorders and Conditions

The invention provides methods for identifying compounds that can modulate CAR-mediated cholesterol regulation. These methods involve testing candidate therapeutic agents to determine whether the agent can modulate CAR-mediated regulation of cholesterol levels. A compound “modulates” CAR-mediated regulation of cholesterol levels if administration of that compound to an animal results in a change in cholesterol levels in the animal. In a presently preferred embodiment, the change is a decrease in cholesterol levels.

In presently preferred embodiments, the candidate therapeutic agents are pre-screened in one or more in vitro assays to identify those compounds that can bind to a CAR polypeptide and/or can modulate CAR-mediated regulation of gene expression.

A. Pre-screening in vitro to Identify Candidate Therapeutic Agents for Use in Treating CAR-mediated Disorders and Conditions

In a presently preferred embodiment, potential candidate therapeutic agents are screened in vitro before in vivo testing. If the candidate therapeutic agent is active in an in vitro screening assay, then the candidate therapeutic agent is more likely to affect CAR-mediated gene expression in vivo. CAR polypeptides for use in the assays can be prepared using methods that are known in the art. Nucleic acids that encode CAR are described in, for example, WO 96/36230; U.S. Pat. Nos. 5,686,574, 5,710,017, and 5,756,448, Baes et al., supra, Forman et al., supra, and Choi et al, supra. Also, chimeric polypeptides that include a CAR ligand binding domain can be expressed as a fusion protein with a domain that permits facile purification (e.g., glutathione S-transferase). Polynucleotide vectors that facilitate the expression of fusion proteins are commercially available (e.g., New England Biolabs, Invitrogen and Novagen). For example, the glutathione S-transferase-CAR ligand binding domain protein can be expressed in E. coli and purified over a glutathione agarose column (Smith and Johnson (1988) Gene 67: 31-40; Guan and Dixon (1991) Anal. Biochem. 192: 262-267; Ausubel et al., supra. Other fusion partners, such as a poly-histidine tag and the maltose binding protein (New England Biolabs), are well known in the art and can be used to express a CAR fusion protein.

1. Direct and Displacement Assays

Candidate therapeutic agents can be pre-screened for their ability to interact directly with a polypeptide that includes a CAR ligand binding domain (e.g., a full-length CAR polypeptide or a fusion protein), or with a CAR ligand.

One type of assay that can be used is a direct binding assay, which measures the amount of candidate therapeutic agent that can bind to a CAR polypeptide or to a polypeptide that has a CAR ligand binding domain. Another type of assay that can be used to pre-screen candidate therapeutic agents is to carry out a direct binding assay with a labeled CAR ligand, such as labeled 5α-androst-16-en-3α-ol, 5α-androst-16-en-3α-ol acetate, 5α-androstane-3α-ol, 5α-androst-16-en-3α-ol acetate or 5β-pregnan-3,20-dione, in the presence of a candidate therapeutic agent. A candidate therapeutic agent that decreases the amount of labeled CAR ligand that is bound to a CAR polypeptide or a polypeptide that has a CAR ligand binding domain, is of interest for future screening for its ability to modulate cholesterol levels in vivo.

These assays can be carried out using labeled candidate therapeutic agents which are then incubated with a polypeptide that has a CAR ligand binding domain (e.g., a full-length CAR polypeptide or a fusion protein). Labels include radioisotopes, immunochemicals, fluorophores, and the like. Those of skill in the art will recognize a variety of ways of separating the bound labeled candidate therapeutic agent from the free labeled candidate therapeutic agent. The affinity of the labeled candidate therapeutic agent for a CAR polypeptide can be calculated using standard ligand binding methods.

Alternatively, an assay such as the fluorescence polarization assay or the fluorescence resonance energy transfer assay can be employed to identify candidate therapeutic agents. These assays do not require the separation of bound and free labeled candidate therapeutic agent. Fluorescence polarization (FP) or fluorescence anisotropy is a useful tool for the study of molecular interactions (see, e.g. http://www.panvera.com/tech/appguide/fpintro.html, Nov. 4, 1999). First, a molecule labeled with a fluorophore is excited with plane polarized light. If the fluorescent molecule stays stationary while in the excited state, light is emitted in the same polarized plane. If the excited fluorescently labeled molecule rotates out of the plane of the polarized light while in the excited state, light is emitted from the molecule in a different plane. For example, if vertical polarized light is used to excite the fluorphore, the emission spectra can be monitored in the vertical and horizontal planes. Fluorescence polarization is calculated as shown in the following formula:

Fluorescent polarization=P=(Int ∥−Int^{⊥})/(Int ∥+Int^{⊥}).

Int ∥ is the intensity of the emission parallel to the excitation plane. Int^{⊥} is the intensity of the emission perpendicular to the excitation plane.

A small fluorescently labeled molecule, when free in solution, can emit depolarized light when excited with the proper wavelength of light. If, however, the molecule (e.g., a ligand) binds to a second molecule (e.g. a receptor) the fluorescently labeled molecule is more constrained so the light emitted is more polarized and the fluorescence polarization (FP) value is higher. Thus, a higher FP value indicates that the fluorescently labeled molecule is able to bind to the second molecule. A competition assay also can be performed using FP. If an unlabeled molecule is present in the solution, then it will compete for binding to the second molecule, e.g., the antibody and the FP value will be decreased. Thus, FP can be used in competitive assays.

Commercial assays exist to test the affinity of test compounds for human estrogen receptor α and β using a fluorescently labeled estrogen compound (see, Panvera, Madison, Wis., publications Lit.#'s L0069, L0082, L0084, L0095, L0072, L0085). Similarly, candidate therapeutic agents can be fluorescently labeled with a fluorophore that is active in a FP assay. For example, N-terminal amines of proteins, peptide, or peptide analogs can be labeled with fluorescein (Panvera, publications Lit. # L0057 and L0059) or a small fluorescent compound. Briefly, a fluorescein-C₆-succinimidyl ester can be conjugated to peptides or proteins. The fluorescein labeled peptide/protein can then be purified from the unreacted fluorescein-C₆-succinimidyl ester using thin-layer chromatography or gel filtration chromatography. If the labeled candidate therapeutic compound can bind to a polypeptide that has a CAR ligand binding domain, the level of polarization is increased. The FP assay can be used to assay the ability of a fluorescently labeled CAR ligand to bind to a CAR polypeptide.

Alternatively, a candidate therapeutic agent can be screened for its ability to decrease the FP of a fluorescently labeled known CAR ligand complexed with a CAR polypeptide or a polypeptide comprising a CAR ligand binding domain. Briefly, a known CAR ligand, such as 5α-androst-16-en-3α-ol, 5α-androst-16-en-3α-ol acetate, 5α-androstane-3α-ol, 5α-androst-16-en-3α-ol acetate or 5β-pregnan-3,20-dione can be labeled with a fluorescent moiety. A candidate therapeutic agent has affinity for the CAR ligand binding domain if it can decrease the FP value of the fluorescently labeled CAR ligand and CAR; the candidate therapeutic agent is displacing or inhibiting the ability of the fluorescently labeled CAR ligand to bind to the ligand binding domain of CAR.

Methods employing the technique of fluorescence resonance energy transfer (FRET) can be employed using the methods and compositions of the present invention. FRET occurs between two fluorophores when the excitation of the donor fluorophore is transferred to the acceptor fluorophore. This interaction is dependent on the distance between the donor and acceptor fluorophore and distance-dependent interaction between a donor and acceptor molecule. The donor and acceptor molecules are fluorophores. If the fluorophores have excitation and emission spectra that overlap, then in close proximity (typically around 10-100 angstroms) the excitation of the donor fluorophore is transferred to the acceptor fluorophore. The relative proximity of the first and second labels is determined by measuring a change in the intrinsic fluorescence of the first or second label. Commonly, the emission of the first label is quenched by proximity of the second label.

Many appropriate interactive labels for FRET are known. For example, fluorescent labels, dyes, enzymatic labels, and antibody labels are all appropriate. Examples of preferred interactive fluorescent label pairs include terbium chelate and TRITC (tetrarhodamine isothiocyanate), europium cryptate and allophycocyanin and many others known to one of skill. Similarly, two colorimetric labels can result in combinations that yield a third color, e.g., a blue emission in proximity to a yellow emission produces an observed green emission.

With regard to preferred fluorescent pairs, there are a number of fluorophores which are known to quench each other. Fluorescence quenching is a bimolecular process that reduces the fluorescence quantum yield, typically without changing the fluorescence emission spectrum. Quenching can result from transient excited interactions, (collisional quenching) or, e.g., from the formation of nonfluorescent ground state species. Self-quenching is the quenching of one fluorophore by another; it tends to occur when high concentrations, labeling densities, or proximity of labels occurs. Some excited fluorophores interact to form excimers, which are excited state dimers that exhibit altered emission spectra (e.g., phospholipid analogs with pyrene sn-2 acyl chains); See Haugland (1996) Handbook of Fluorescent Probes and Research Chemicals, published by Molecular Probes, Inc., Eugene, Oreg.

The Forster radius (Ro) is the distance between fluorescent pairs at which energy transfer is 50% efficient (i.e., at which 50% of excited donors are deactivated by FRET). The magnitude of R₀ is dependent on the spectral properties of donor and acceptor dyes: R₀=[8.8×10²³·K²·n⁻⁴·QY_{D}·J(λ)]^{⅙} Å; where K²=dipole orientation range factor (range 0 to 4, K²=⅔ for randomly oriented donors and acceptors).; QY_{D}=fluorescence quantum yield of the donor in the absence of the acceptor; n=refractive index; and J(λ)=spectral overlap integral=∫ε_{A}(λ)·F_{D}·(λ4)dλ cm³M⁻¹, where ε_{A}=extinction coefficient of acceptor and FD=fluorescence emission intensity of donor as a fraction of total integrated intensity. Some typical Ro are listed for typical donor acceptor pairs:

| Donor | Acceptor | Rₒ (Å) |
|---|---|---|
| Fluorescein | Tetramethylrhodamine | 55 |
| IAEDANS | Fluorescein | 46 |
| EDANS | DAB CYL | 33 |
| BODIPY FL | BODIPY FL | 57 |
| Fluorescein | QSY-7 dye | 61 |

An extensive compilation of Ro values are found in the literature; see Haugland (1996), supra. In most uses, the donor and acceptor dyes are different, in which case FRET can be detected by the appearance of sensitized fluorescence of the acceptor or by quenching of the donor fluorescence. When the donor and acceptor are the same, FRET is detected by the resulting fluorescence depolarization.

In addition to quenching between fluorophores, individual fluorophores are also quenched by nitroxide-labeled molecules such as fatty acids. Spin labels such as nitroxides are also useful in the liquid phase assays of the invention.

Candidate therapeutic agents and a polypeptide comprising a CAR ligand binding domain can be labeled with FRET pairs. If the candidate therapeutic agent can directly interact with the polypeptide comprising a CAR ligand binding domain then fluorescence resonance energy transfer can take place and the affinity can be measured. Alternatively, a known CAR ligand, such as 5α-androst-16-en-3α-ol, 5α-androst-16-en-3α-ol acetate, 5α-androstane-3α-ol, 5α-androst-16-en-3α-ol acetate or 5β-pregnan-3,20-dione can be labeled with an appropriate FRET label and incubated with an FRET fluorophore labeled polypeptide comprising a CAR ligand binding domain. Fluorescence resonance energy transfer can take place between the labeled CAR ligand and the labeled polypeptide comprising a CAR ligand binding domain. If a candidate therapeutic agent were incubated with the complex, then the amount of FRET would be lowered if the candidate therapeutic agent can inhibit or displace the binding of the labeled CAR ligand to the polypeptide comprising a CAR ligand binding domain.

Additional methods of identifying ligands for nuclear receptors such as the CAR receptors can be adapted from assays described in WO 99/27365. Briefly, these assays use a peptide sensor to which is attached a detectable label. The peptides are based on corepressor or coactivator protein motif sequences, either naturally occurring or derived from mutational analysis. Alternatively, the peptides can be obtained through randomizing residues and selecting for binding to the CAR receptor polypeptide. In some embodiments, panels of predetermined or randomized candidate sensors are screened for receptor binding.

In typical embodiments, the sensor peptides are labeled with a detectable label. The detectable labels can be primary labels (where the label comprises an element that is detected directly or that produces a directly detectable element) or secondary labels (where the detected label binds to a primary label, as is common in immunological labeling). An introduction to labels, labeling procedures and detection of labels is found in Polak and Van Noorden (1997) Introduction to Immunocytochemistry, 2nd ed., Springer Verlag, NY and in Haugland (1996) Handbook of Fluorescent Probes and Research Chemicals, a combined handbook and catalogue published by Molecular Probes, Inc., Eugene, Oreg. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral colorimetric labels such as colloidal gold or colored glass or plastic (e.g. polystyrene, polypropylene, latex, etc.) beads. The label may be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions. In a presently preferred embodiment, the detectable label is a fluorescent label, in which case fluorescence polarization detection provides a sensitive and efficient means of detecting whether the peptide sensor is bound to the CAR receptor polypeptide. See, e.g., Schindler et al. (1995) Immunity 2: 689-697).

The sensor polypeptide and the CAR polypeptide are incubated under conditions that are suitable for sensor binding to the receptor polypeptide. In some embodiments, a candidate modulator of CAR binding to a corepressor or coactivator is included in the reaction mixture. If a candidate modulator decreases binding of the sensor peptide to the CAR polypeptide, the candidate modulator is a potential lead compound for blocking the CAR-mediated effect on transcription.

2. Assays for Modulators of the Transcriptional Activity of CAR

This application describes for the first time, an agonist for the CAR receptor—5β-pregnan-3,20-dione. Previously, there are were known agonists for the CAR receptors. That is, there are were no compounds known to increase the transcriptional activity of CAR or to diminish the ability of a CAR inverse agonist (e.g., 5α-androst-16-en-3α-ol, 5α-androst-16-en-3α-ol acetate, 5α-androstane-3α-ol, 5α-androst-16-en-3α-ol acetate) to inhibit the transcriptional activity of CAR. Methods for discovering such agonists are described herein. Also, provided by the invention are methods for pre-screening candidate therapeutic agents as in a reporter gene assay.

The methods can involve the use of a full-length CAR polypeptide or a chimeric polypeptide that includes a CAR ligand binding domain that is linked to a DNA binding domain for which a known response element is available. Suitable chimeric polypeptides and methods for their production are described herein. Methods for expressing CAR polypeptides and carrying out gene transcription assays are described in WO 96/36230; U.S. Pat. Nos. 5,686,574, 5,710,017, and 5,756,448, Baes et al., supra, Forman et al., supra, Choi et al., supra.

The response element that is recognized by the DNA binding domain used in the chimeric polypeptide is generally contained in the reporter gene construct. The response element is part of the promoter that is linked to the reporter gene. Response elements, including glucocorticoid response elements (GRE) and estrogen response elements (ERE), are described in, for example, Jantzen et al. (1987) Cell 49: 29; Martinez et al. (1987) EMBO J. 6: 3719 and Burch et al. (1988) Mol. Cell. Biol. 8: 1123. Many other response elements are known; a commonly used response element is the GAL4 upstream activating sequence (UAS_{G}) (Keegan et al. (1986) Science 14: 699-704), which is responsive to binding by chimeric receptors that include the GAL4 DNA binding domain. Response elements that contain the DR-5 elements such as those from retinoic acid response elements (RAREs) that exist in the human retinoic acid receptor beta (RARβ) gene and the human alcohol dehydrogenase 3 (ADH3) gene are also suitable for use in a gene activation assay, provided the DNA binding domain is from a CAR polypeptide. DR-4 elements in the CYP2B6 gene also act as CAR response elements ( Sueyoshi et al., supra).

In presently preferred embodiments, the promoter and response element are operably linked to a reporter gene that, when expressed, produces a readily detectable product. A variety of reporter gene plasmid systems are known, such as the chloramphenicol acetyltransferase (CAT) and β-galactosidase (e.g., bacterial LacZ gene) reporter systems, the firefly luciferase gene (See, e.g., Cara et al., (1996) J. Biol. Chem., 271: 5393-5397), the green fluorescence protein (see, e.g., Chalfie et al. (1994) Science 263:802) and many others. Examples of reporter plasmids are also described in U.S. Pat. No. 5,071,773. Selectable markers which facilitate cloning of the vectors of the invention are optionally included. Sambrook and Ausubel, both supra, provide an overview of selectable markers.

In some embodiments of this assay, the reporter plasmid and an expression plasmid that directs expression of the chimeric receptor are introduced into a suitable host cell. Standard transfection methods can be used to introduce the vectors into the host cells. For mammalian host cells, preferred transfection methods include, for example, calcium phosphate precipitation (Chen and Okayama (1988) BioTechniques 6: 632), DEAE-dextran, and cationic lipid-mediated transfection (e.g., Lipofectin) (see, e.g., Ausubel, supra.).

The cell containing the chimeric receptor is contacted with a candidate therapeutic agent. For example, a cell that contains a reporter gene construct and the chimeric peptide can be grown in the presence and absence of a candidate therapeutic agent. Due to the constitutive transcriptional activity of CAR, cells grown in the absence of a candidate therapeutic agent will exhibit reporter gene expression. If the candidate therapeutic agent is a CAR agonist then the reporter gene activity will be increased over basal CAR activity. On the other hand, if the candidate therapeutic agent is a CAR antagonist then the reporter gene activity will be decreased as compared to basal CAR transcriptional activity. The compounds are examples of CAR antagonists. Alternatively, cells can be contacted with a candidate therapeutic agent in the presence and absence of a CAR inverse agonist. The cells grown in the presence of the CAR inverse agonist alone should exhibit a lower level of reporter gene expression due to the repression of CAR's basal activity. If the candidate therapeutic agent is an agonist, however, then the repression of reporter gene activity by the antagonist should be decreased (reporter gene expression should increase as compared to the treated cells treated only with the antagonist).

In addition, potential agonists for the CAR receptors of the invention can include other receptor polypeptides, coactivators, and the like, which comprise the cellular machinery for regulation of gene expression. For example, nuclear hormone receptors often interact with transcriptional coactivators. Thus, the invention also provides methods of identifying coactivators, corepressors and other molecules that interact with CAR receptors. These assay methods can involve introducing a coactivator or a corepressor that is a candidate therapeutic agent for CAR receptors into a host cell that contains a chimeric CAR receptor and reporter plasmid. The coactivator can be introduced by means of an expression construct; this expression construct can be present on the same or a different vector than the expression construct for the chimeric receptor.

a. Cells.

Those of skill in the art will recognize a wide variety of cells that may be used in the methods of the present invention. The cells may be, but are not limited to, primary cultures of cells, transformed cells, neoplastic cells, and nontransformed cells. See WO 96/36230 for examples of cells that can be used in the gene expression assay. Cell lines that may be used in the gene expression assay include liver cell lines (e.g., Hep-G2), primary hepatocytes, adipocyte or pre-adipocyte cell lines (e.g., 3T3-L1 cells, 3T3-L1 cells, 3T3-442-A cells, OB17 cells, and the like, as well as CV-1 cells, HuTu80 cells, F9 cells, NTERA2 cells, NB4 cells, HL-60 cells, HEK 293 cells, HeLa cells, NIH-3T3 cells and the like. See WO 96/36230. In preferred embodiments of the present invention CV-1 and COS cells are used.

b. CAR Ligand Binding Domain-receptor DNA Binding Domain Fusions

The chimeric receptors of the invention include those having a ligand binding domain that is at least substantially identical to a ligand binding domain of a CAR, as well as those that have a DNA binding domain that is not substantially identical to a DNA binding domain of an CAR. For example, the DNA binding domain binding domain can be about 90% or less identical to that of a CAR polypeptide, more preferably about 75% or less, and most preferably about 60% or less identical. Often, the DNA binding domain is derived from a receptor other than a CAR receptor. In a typical embodiment, the DNA binding domain is at least substantially identical to a DNA binding domain from a nuclear hormone receptor, a steroid hormone receptor, or the yeast transcription factor GAL4.

The modular nature of transcription regulators facilitates the construction of chimeric receptors that have domains that are derived from different receptors (Green and Chambon (1986) Nature 324: 615-617). For example, DNA binding domains derived from steroid, thyroid, and retinoid hormone receptor are suitable for use in the chimeric receptors of the invention. The DNA binding domains of receptors for steroid, thyroid, and retinoid hormones typically include two zinc finger units (Rhodes and Klug (February 1993) Scientific American, pp. 56-65). The DNA binding domains of these receptors, as are those of the CAR receptors, are generally cysteine-rich regions of about 65 amino acids that fold into two cysteine-rich “C4” type zinc fingers. The boundaries for many DNA binding domains have been identified and characterized for the steroid hormone superfamily. See, e.g., Giguere et al. (1986) Cell 46:645-652; Hollenberg et al. (1987) Cell 49:39-46; Green and Chambon (1987) Nature 325:74-78; Miesfield et al. (1987) Science 236:423-427; and Evans (1988) Science 240:889-895.

Examples of receptors from which one can derive DNA binding domains that are suitable for use in the chimeric receptors of the invention include, for example, androgen receptors, estrogen receptors, glucocorticoid receptors, mineralcorticoid receptors, progesterone receptors, retinoic acid receptors (including α, β (hap), and γ), thyroid hormone receptors (including α and β), the gene product of the avian erythroblastosis virus oncogene v-erbA (which is derived from a cellular thyroid hormone receptor), vitamin D3 receptor, Drosophila ecdysone receptor (EcR), COUP transcription factor (also known as ear3) and its Drosophila homolog 7UP (svp), hepatocyte nuclear factor 4 (HNF-4), Ad4BP, apolipoprotein AI regulatory protein-1 (ARP-1), peroxisome proliferator activated receptor (PPAR), Drosophila protein knirps (kni), Drosophila protein ultraspiracle (usp; chorion factor 1), human estrogen receptor related genes 1 and 2 (err1 and err2), human erbB related gene 2 (ear2), human NAK1/mouse nur/77 (N10)/rat NGFI-B; Drosophila protein embryonic gonad (egon), Drosophila knirps-related protein (knr1), Drosophila protein tailless (tll), Drosophila 20-O-ecdysone regulated protein E75, and Drosophila Dhr3. Some of these and other suitable receptors are described in, for example, Evans, R M (1988) Science 240: 889-895; Gehrig, U. (1987) Trends Biochem. Sci. 12: 399-402; Beato, M. (1989) Cell 56: 335-344; Laudet et al. (1992)EMBO J. 11: 1003-1013.

In some embodiments, the chimeric receptors of the invention include a DNA binding domain from a DNA-binding polypeptide other than a nuclear receptor. For example, chimeric receptors that have the DNA binding domain of GAL4, which is a positive regulatory protein of yeast (Giniger et al. (1985) Cell 40: 767-774; Sadowski et al. (1992) Gene 118: 137-141) linked to a ligand binding domain of a CAR polypeptide are provided. GAL4 DNA binding domain-containing fusion proteins can be readily expressed by cloning a coding sequence for a CAR ligand binding domain into a commercially available expression vector that includes a GAL4 DNA binding domain coding sequence under the control of a promoter (e.g., pAS2-1 (CLONTECH Laboratories, Inc.). Another example of a well-characterized DNA binding domain for which expression vectors are commercially available is that of LexA (pLexA, CLONTECH). The chimeric receptors can also include a nuclear localization sequence associated with the DNA binding domain (see, e.g., Silver et al. (1984) Proc. Nat'l. Acad. Sci. USA 81: 5951-5955 for a GAL4 nuclear localization sequence).

The chimeric receptors of the invention can use an entire molecule as a DNA binding domain, or can use portions of molecules that are capable of binding to nucleic acids, directly or indirectly. To identify such DNA binding domains, one can perform assays such as an electrophoretic mobility shift assay (EMSA) (Scott et al. (1994) J. Biol. Chem. 269: 19848-19858), in which a nucleic acid of interest is allowed to associate with various fragments of a polypeptide to identify those fragments that are capable of binding to the nucleic acid. Association of a portion of the protein with the nucleic acid will result in a retardation of the electrophoretic mobility of the nucleic acid. Another method by which one can identify DNA binding moieties that are suitable for use as DNA binding domains is DNase I footprinting, which is well known to those of skill in the art.

The DNA binding domain can be either a polypeptide or a nucleic acid. Where the DNA binding domain is a nucleic acid, the nucleic acid will be capable of specifically hybridizing to a target nucleic acid site, such as a response element. Hybridization of the nucleic acid to the target site will place the chimeric receptor in a position suitable for activating or repressing expression of a gene that is linked to the target site. An example of an oligonucleotide being chemically linked to a protein by chemical coupling is found in Corey et al. (1989) Biochemistry 28: 8277-8286.

These chimeric receptors are useful, for example, in assays to identify modulators of CAR transcriptional regulation activity as described below.

C. Production of Chimeric CAR Receptors

To form a chimeric receptor of the invention, the ligand binding domain and the DNA binding domain are linked together. Suitable methods of forming such linkages are known to those of skill in the art. For a review of methods for constructing fusion proteins between receptor ligand binding domains and DNA binding domains, see, e.g., Mattioni et al. (1994) Methods in Cell Biology 43(Pt A): 335-352. The linkage can be done using either recombinant or chemical methods. For example, a cysteine residue can be placed at either end of a domain so that the domain can be linked to another domain by, for example, a sulfide linkage. More typically, the ligand binding domains and DNA binding domains are joined by linkers, which are typically polypeptide sequences, such as poly-glycine sequences of between about 5 and 200 amino acids, with between about 10-100 amino acids being typical. In some embodiments, proline residues are incorporated into the linker to prevent the formation of significant secondary structural elements by the linker. Preferred linkers are often flexible amino acid subsequences which are synthesized as part of a recombinant fusion protein. In one embodiment, the flexible linker is an amino acid subsequence comprising a proline such as Gly(x)-Pro-Gly(x) where x is a number between about 3 and about 100. A linker can also be a single peptide bond, or one or more amino acid residues. In other embodiments, a chemical linker is used to connect synthetically or recombinantly produced ligand binding domain and DNA binding domain subsequences. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

The chimeric receptors are conveniently produced by recombinant expression in a host cell. Accordingly, the invention provides chimeric nucleic acids that encode a fusion protein that includes a DNA binding domain and a ligand binding domain, at least one of which is at least substantially identical to the corresponding domain of a CAR nuclear receptor of the invention. In some embodiments, the chimeric nucleic acid will also encode a linker region that provides a link between the two domains. Techniques for making such chimeric nucleic acids are known to those of skilled in the art. For example, recombinant methods can be used (see, e.g., Berger and Sambrook, both supra.). Alternatively, the nucleic acid encoding the chimeric receptors can be synthesized chemically.

To obtain expression of a chimeric receptor, a nucleic acid that encodes the chimeric receptor is generally placed under the control of a promoter and other control elements that can drive expression of the chimeric gene in a desired host cell. Accordingly, the invention also provides expression cassettes in which a promoter and/or other control elements are operably linked to a polynucleotide that encodes a chimeric receptor. Suitable promoters, other control sequences, and expression vectors are described above.

3. Assays for the Effect of a Candidate Therapeutic Agent on the Transcription of a Cholesterol Regulated Gene.

Another method of the present invention involves assaying for the amount of mRNA of a gene involved in cholesterol biosynthesis or regulation. A “mRNA of a gene involved in the regulation of cholesterol levels” is a polynucleotide transcribed from a gene that functions in maintaining cholesterol homeostasis. Examples of such a gene include, without limitation, genes that encode 3-hydroxy-3-methylglutaryl CoA (HMG CoA) synthase, HMG CoA reductase, squalene synthase, acetyl-CoA carboxylase, fatty acid synthase, stearoyl-CoA desaturase-1, and the LDL receptor.

Briefly, cells are incubated in the presence and absence of a candidate therapeutic agent. After the incubation, RNA from the cells is isolated. The presence of the mRNA of a cholesterol regulated gene can then be detected through methods known to those of skill in the art. For example, one can detect the RNA through reverse transcription followed by PCR. Alternatively, the RNA can be electrophoresed, blotted, and hybridized with an appropriate probe directed towards the gene of interest. Other methods, such as RNAse protection, are known in the art for detecting RNA sequences of interest. If the candidate therapeutic agent is a CAR agonist then the cholesterol levels should decrease and the transcription of cholesterol regulated genes would be affected accordingly. Similarly, if the candidate therapeutic agent is a CAR antagonist then the cholesterol levels should increase and the transcription of cholesterol regulated genes would be affected accordingly.

4. Candidate Therapeutic Agents.

A “candidate therapeutic agent” is a compound that is being tested for its usefulness in the treatment of a CAR-mediated disorder or condition, or is being tested in a ligand binding assay or a gene activation assay. The candidate therapeutic agent can be a naturally occurring compound, one that is artificially synthesized, or one that is made by a combination these methods. Essentially any chemical compound can be pre-screened as a CAR candidate therapeutic agent in the assays of the invention. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like. Examples of candidate therapeutic agents include 5β-pregnan-3,20-dione, TCPOBOP, and the compounds listed in Table 1 (T0438248 (AsInEx Ltd., Moscow, Russia), T0434523 (Bionet Research, Camelford, UK), T0318784 (ChemBridge Corporation, San Diego, Calif.), T0037566 (Aldrich Corporation), T0509562 (AsInEx Ltd., Moscow, Russia)).

TABLE 1

| | Empirical | Molecular | |
|---|---|---|---|
| Compound | formula | Weight (Da) | Structure |
| T0438248 | C₃₄H₃₉Cl₂NO₄ | 696.591 | 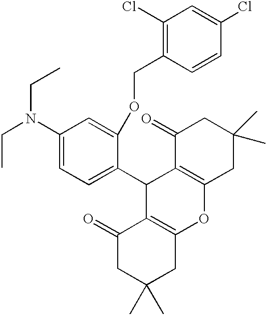 |
| T0434523 | C₂₃H₁₆Cl₃N₃S₂ | 504.891 | 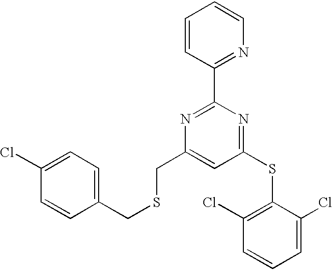 |
| T0318784 | C₂₃H₂₀Cl₂N₂O₂ | 427.329 | 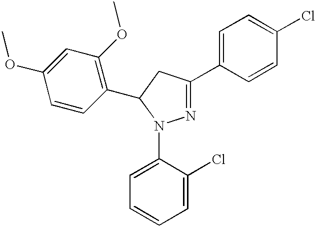 |
| T0037566 | C₂₃H₂₃NO₃ | 361.439 | 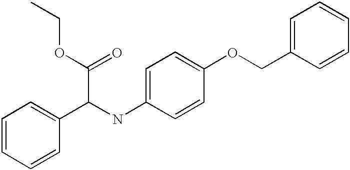 |
| T0509562 | C₂₄H₂₇NO₅ | 409.479 | 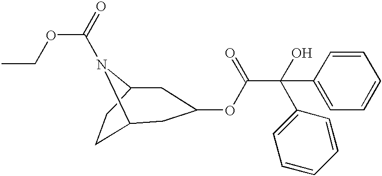 |

A candidate therapeutic agent “modulates” CAR if the candidate therapeutic agent increases or decreases the ability of a CAR polypeptide to act in a selected system. Such selected systems include, but are not limited to: increasing or decreasing the amount of serum cholesterol, gene activation assays, decreasing serum cholesterol in a mammal with two wild-type genes as compared to a CAR compromised mammal, or to affect the ability of CAR to bind to a ligand. Subjects or assays that are treated with a candidate therapeutic agent are compared to control samples without the candidate therapeutic agent, to examine the extent of inhibition or activation of CAR-mediated regulation of cholesterol levels or CAR-mediated gene activation, or CAR ligand binding to CAR, in an assay. Control samples (untreated with a candidate therapeutic agent) are assigned a relative CAR activity value of 100. Inhibition of CAR activity is achieved when the CAR activity value of the candidate therapeutic agent sample relative to the control is about 75, preferably 50, more preferably 25. Activation is achieved when the CAR activity value of the test sample relative to the control is about 150, more preferably 200.

In some preferred embodiments, high throughput screening methods are used to test a combinatorial library that contains a large number of potential therapeutic compounds (potential modulator compounds). In some embodiments, compounds that can be dissolved in aqueous or organic (e.g., DMSO-based) solutions are used. The assays are designed to screen large chemical libraries by automating the assay steps and providing compounds from any convenient source to assays, which are typically run in parallel (e.g., in microtiter formats on microtiter plates in robotic assays). “Combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., AsInEx, Moscow, Russia, Tripos, Inc., St. Louis, Mo., ChemStar, Ltd, Moscow, RU, 3D Pharmaceuticals, Exton, Pa., Martek Biosciences, Columbia, Md., etc.).

C. In vivo testing of candidate therapeutic agents

The term “administering” refers to the method of contacting a compound with the subject. Modes of “administering,” may include but are not limited to, methods that involve contacting the compound intravenously, intraperitoneally, intranasally, transdermally, topically, via implantation, subcutaneously, parentally, intramuscularly, orally, systemically, and via adsorption. The candidate therapeutic agent can be formulated as a pharmaceutical composition in the form of a syrup, an elixir, a suspension, a powder, a granule, a tablet, a capsule, a lozenge, a troche, an aqueous solution, a cream, an ointment, a lotion, a gel, or an emulsion.

Several embodiments of methods can be used to test the efficacy of a candidate therapeutic agent to modulate CAR-mediated regulation of cholesterol levels. Preferably, the candidate therapeutic agent will act as an agonist and lower the level of cholesterol in a CAR-mediated process or block the action of a CAR antagonist.

1. Administration of a Candidate Therapeutic Agent and a CAR Antagonist to a Mammal.

In this method, a composition comprising a candidate therapeutic agent and a CAR antagonist, such as 5α-androst-16-en-3α-ol, 5α-androst-16-en-3α-ol acetate, 5α-androstane-3α-ol, 5α-androst-16-en-3α-ol acetate are administered to a first mammal. A second mammal is administered a composition comprising the same CAR antagonist. If the level of cholesterol in the candidate therapeutic agent/antagonist treated mammal is lower than in the antagonist treated mammal, then the candidate therapeutic agent is suitable for use in treating a CAR-mediated disorder or condition.

2. Administration of a Candidate Therapeutic Agent to a Cholesterol Elevated Mammal.

In this method, a composition comprising a candidate therapeutic agent is administered to a mammal with an elevated level of cholesterol. If the candidate therapeutic agent treated mammal exhibits a lower level of cholesterol, then the candidate therapeutic agent has been identified for use in treating a CAR-mediated disorder or condition.

3. Administration of a Candidate Therapeutic Agent to a CAR Compromised and a CAR Non-compromised Mammal.

In this method, a candidate therapeutic agent is administered to a CAR compromised mammal and to a CAR non-compromised mammal. An example of a CAR compromised mammal is a mammal with a disruption in at least one CAR allele that prevents a functional CAR polypeptide from being produced. A male mammal heterozygous for a CAR disruption or a mammal with a disruption in both CAR alleles is also an example of a CAR compromised mammal. A CAR non-compromised mammal is a mammal that has at least one allele that produces a functional CAR polypeptide. The candidate therapeutic agent will be useful in the treatment of a CAR-mediated disorder or condition if the level of cholesterol is lower in the CAR non-compromised mammal as compared to the CAR compromised mammal.

4. Cholesterol Assays.

Assays for the level of cholesterol are well known in the art and are commercially available (e.g., Sigma Chemical Company's enzymatic assay for cholesterol, Catalog no. 353-20). The serum from a mammal exposed to a candidate therapeutic agent can be analyzed for cholesterol. Blood is withdrawn from the subject mammal(s) and is allowed to clot. The serum sample is obtained through low-speed centrifugation of the clotted blood sample. The serum can then be analyzed through a variety of methods known to those in the art for cholesterol, HDL cholesterol, LDL cholesterol and VLDL cholesterol.

Serum cholesterol assays are commercially available from vendors such as Sigma Chemical Company and Boehringer Mannheim. For example, the Boehringer Mannheim Diagnostics high performance cholesterol assay is a colorimetric cholesterol assay. Briefly, the cholesterol in the serum is oxidized to produce cholest-4-en-3-one and hydrogen peroxide. The hydrogen peroxide is incubated with phenol, 4-aiminophenazone, and peroxidase. This reaction produces a p-quinone imine dye, which can be detected in a spectrophotometer at 500 nm. Using known amounts of cholesterol, a standard curve can be constructed to determine the concentration of cholesterol in serum or a serum-derived sample. A Sigma Chemical Company total cholesterol assay relies on a different enzymatic assay. See Sigma catalog no. 352-20. The serum is incubated with cholesterol esterase and cholesterol oxidase to oxidize cholesterol and cholesterol esters to produce cholest-4-en-3-one and hydrogen peroxide. The hydrogen peroxide is reacted with 4-aminoantipyrine, p-hydroxybenzene sulfonate, and peroxidase to produce a quinoneimine dye. The presence of the quinoneimine dye can be determined using a spectrophotometer.

The levels of HDL cholesterol, LDL cholesterol, and VLDL cholesterol can also be analyzed. Methods exist in the art for the fractionation and detection of the levels of lipoprotein cholesterol levels. For example, a HDL cholesterol method and a LDL cholesterol method are also available from the Sigma Chemical Company. For the HDL cholesterol assay, the LDL and VLDL lipoproteins are first precipitated. Then, sample is centrifuged and the VLDL and LDL precipitates are removed. The HDL remains soluble and the HDL cholesterol can be assayed through Sigma Procedure no. 352. For the LDL cholesterol assay, the serum sample is incubated with antisera coated latex beads. The antisera are directed against the high-density lipoprotein (HDL) and the very low-density lipoprotein (VLDL). The solution is filtered from the beads. The filtrate contains only LDL and thus only LDL cholesterol. The solution can then be assayed for LDL cholesterol using the Sigma enzymatic method. Alternatively, the distribution of cholesterol among lipoprotein particles can be determined by FPLC using a Sepharose 4B column for particle size exclusion. The cholesterol content of the fractions are determined using a Hitachi clinical analyzer.

5. Assays for the Level of mRNA of a Cholesterol Regulated Gene.

RNA can be extracted from cells or animals treated with a candidate therapeutic agent. The RNA samples can then be analyzed to ascertain if the level of a mRNA regulated by cholesterol has been increased or decreased by the administration of the candidate therapeutic agent. See above for examples of cholesterol regulated genes and methods for analyzing RNA.

III. Methods for Treating or Preventing Car-mediated Disorders and Conditions.

CAR-mediated disorders and conditions, such as atherosclerosis, cardiovascular disorders, lipid disorders and hypercholesterolemia, can be treated with therapeutic agent(s) identified using the methods described herein. The candidate therapeutic agents can be pre-screened using the methods described herein. The candidate therapeutic agent is prepared as a pharmaceutical composition and is administered to a subject suffering from a CAR-mediated disorder or condition.

A. Pharmaceutical Compositions.

Accordingly, the present invention provides pharmaceutical compositions comprising a pharmaceutically acceptable carrier or excipient and a candidate therapeutic agent. Candidate therapeutic agents which possess EC₅₀'s for increasing CAR reporter gene activity or repressing a CAR agonist of about 30 μM or less will be particularly useful in the present compositions. More preferably, the candidate therapeutic agent will have an EC₅₀ of about 1 μM or less. Most preferably the compounds will have an EC₅₀ of about 0.01 μM or less. Preferably, the candidate therapeutic agents will cause total cholesterol levels to decrease about 10%, more preferably a decrease of about 20%, and most preferably a decrease of about 25-45%.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg according to the particular application and the potency of the active component. The composition can, if desired, also contain other compatible therapeutic agents.

B. Treatment Regime Using Candidate Therapeutic Agents

The present invention also provides methods of promoting CAR activity or blocking CAR antagonist activity in a cell. In this aspect, a cell is contacted with an CAR-inhibiting amount of a compound or composition above. A CAR-promoting amount can be readily determined using the assays described briefly above, or alternatively, using the assays in the Examples below. Typically, the amount or concentration of compound required to achieve EC₅₀ will be considered an CAR activating or CAR antagonist inhibiting amount. Candidate therapeutic agents are especially useful in the treatment of hypercholesterolemia. In a preferred embodiment, the candidate therapeutic agents are used to treat human patients with total cholesterol levels greater than about 200 mg/dl and/or with LDL cholesterol levels greater than about 130 mg/dl.

In another aspect, the present invention provides methods of treating conditions modulated by CAR in a host, by administering to the host an effective amount of a compound or composition provided above. In therapeutic applications, the compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally.

A variety of conditions are modulated, at least in part, by CAR, including hypercholesterolemia or other conditions associated with abnormal cholesterol or lipid homeostasis such as atheroscleoris, lipid disorders and cardiovascular disorders. The compounds utilized in the pharmaceutical method of the invention are administered at the initial dosage of about 0.001 mg/kg to about 100 mg/kg daily. A daily dose range of about 0.1 mg/kg to about 10 mg/kg is preferred. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Typically, the host or subject in each of these methods is human, although other animals can also benefit from the foregoing treatments.

IV. Transgenic Animals Having Car-mediated Disorders

Transgenic and chimeric non-human mammals and method for generating them are described below. The mammals are useful for testing potential CAR-mediated disorder treatments, and for studying mechanisms of hypercholesteremia.

Transgenic and chimeric non-human mammals were generated that contain cells that lack at least one allele for CAR. Specifically, mice were made that were homozygous null and heterozygous null for the CARβ allele. These animals are useful for investigating the function of CAR in mammalian physiology and biochemistry. More specifically, these animals are useful for studying the effects of CAR modulating compounds on hypercholesterolemia, other lipid disorders, atherosclerosis and cardiovascular disorders.

A “chimeric animal” includes some cells that lack the functional CAR gene of interest and other cells that do not have the inactivated gene. A “transgenic animal,” in contrast, is made up of cells that have all incorporated the specific modification which renders the CAR gene inactive. While a transgenic animal is capable of transmitting the inactivated CAR gene to its progeny, the ability of a chimeric animal to transmit the mutation depends upon whether the inactivated gene is present in the animal's germ cells. The modifications that inactivate the CAR gene can include, for example, insertions, deletions, or substitutions of one or more nucleotides. The modifications can interfere with transcription of the gene itself, with translation and/or stability of the resulting mRNA, or can cause the gene to encode an inactive CAR polypeptide.

The claimed methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, C A, Ed., Transgenic Animal Technology: A Laboratory Handbook, Academic Press, 1994.

One method of obtaining a transgenic or chimeric animal having an inactivated CAR gene in its genome is to contact fertilized oocytes with a vector that includes a CAR-encoding polynucleotide that is modified to contain an inactivating modification. For some animals, such as mice, fertilization is performed in vivo and fertilized ova are surgically removed. In other animals, particularly bovines, it is preferably to remove ova from live or slaughterhouse animals and fertilize the ova in vitro. See DeBoer et al., WO 91/08216. In vitro fertilization permits the modifications to be introduced into substantially synchronous cells. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoel cavity, typically at the 64 cell stage. If desired, the presence of a desired inactivated CAR gene in the embryo cells can be detected by methods known to those of skill in the art. Methods for culturing fertilized oocytes to the pre-implantation stage are described by Gordon et al. (1984) Methods Enzymol. 101: 414; Hogan et al. Manipulation of the Mouse Embryo: A Laboratory Manual, C.S.H.L. N.Y. (1986) (mouse embryo); Hammer et al. (1985) Nature 315: 680 (rabbit and porcine embryos); Gandolfi et al. (1987) J. Reprod. Fert. 81: 23-28; Rexroad et al. (1988) J. Anim. Sci. 66: 947-953 (ovine embryos) and Eyestone et al. (1989) J. Reprod. Fert. 85: 715-720; Camous et al. (1984) J. Reprod. Fert. 72: 779-785; and Heyman et al. (1987) Theriogenology 27: 5968 (bovine embryos). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals. Chimeric mice and germline transgenic mice can also be created through the use of a vendor (e.g., Deltagen, San Carlos, Calif.).

Alternatively, the modified CAR gene can be introduced into embryonic stem cells (ES). These cells are obtained from preimplantation embryos cultured in vitro. See, e.g., Hooper, M L, Embryonal Stem Cells: Introducing Planned Changes into the Animal Germline (Modem Genetics, v. 1), Int'l. Pub. Distrib., Inc., 1993; Bradley et al. (1984) Nature 309, 255-258.

Transformed ES cells are combined with blastocysts from a nonhuman animal. The ES cells colonize the embryo and in some embryos form the germ line of the resulting chimeric animal. See, Jaenisch (1988) Science 240: 1468-1474. Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature 385: 810-813.

The introduction of the modified CAR gene into recipient cells can be accomplished by methods known to those of skill in the art. For example, the modified gene can be targeted to the wild type CAR locus by homologous recombination.

Alternatively, a recombinase system can be employed to delete all or a portion of a locus of interest. Examples of recombinase systems include, the cre/lox system of bacteriophage P1 (see, e.g., Gu et al. (1994) Science 265: 103-106; Terry et al. (1997) Transgenic Res. 6: 349-356) and the FLP/FRT site specific integration system (see, e.g., Dymecki (1996) Proc. Natl. Acad. Sci. USA 93: 6191-6196). In these systems, sites recognized by the particular recombinase are typically introduced into the genome at a position flanking the portion of the gene that is to be deleted. Introduction of the recombinase into the cells then catalyzes recombination which deletes from the genome the polynucleotide sequence that is flanked by the recombination sites. If desired, one can obtain animals in which only certain cell types lack the CAR gene of interest. See, e.g., Tsien et al. (1996) Cell 87: 1317-26; Brocard et al. (1996) Proc. Natl. Acad. Sci. USA 93: 10887-10890; Wang et al. (1996) Proc. Natl. Acad. Sci. USA 93: 3932-6; Meyers et al. (1998) Nat. Genet. 18: 136-41).

The genotype of a CAR non-human mammal or a targeted ES cell can be ascertained through probing a Southern Blot of a sample of the non-human mammal's DNA. See Ausubel et al In rodents, the DNA sample can be isolated from a portion of the animal's tail.

### EXAMPLES

The following examples are offered to illustrate, but not to limit the present invention.

### Example 1

### Production of Mice with Disruptions in at least One CAR Allele

Mice that contain at a disruption in at least one CAR allele were created through creating disruptions in the endogenous CARβ gene.

Materials and Methods

A. Creation of a CAR Targeting Vector

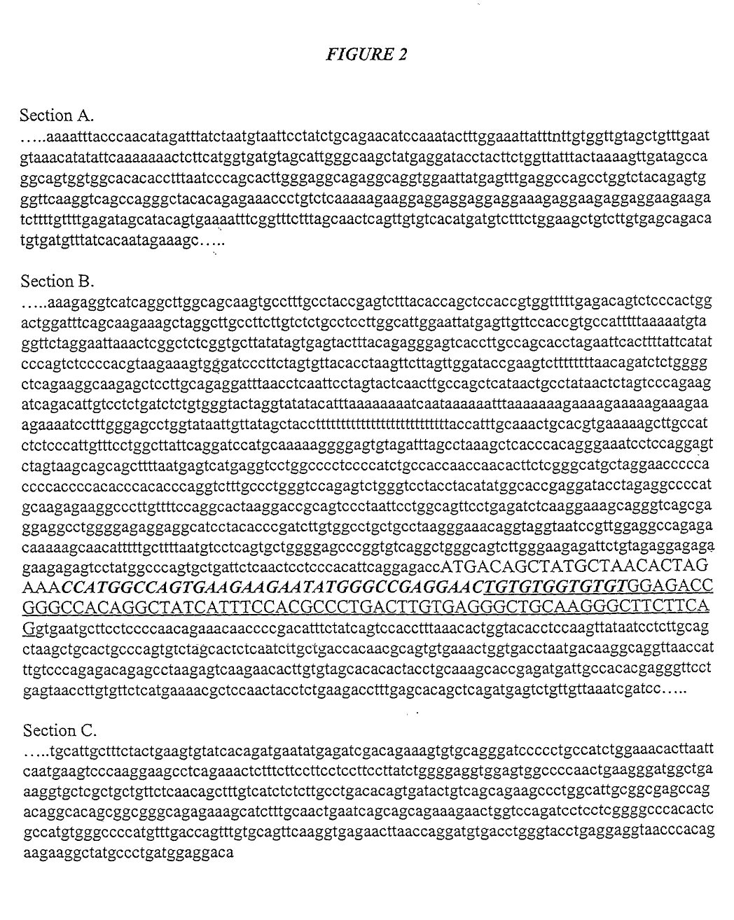

Fragments containing CARβ gene sequences from a mouse 129/Sv genomic clone were inserted into the pGT-N28 gene targeting vector (New England Biolabs, Beverly Mass.). The left arm of the targeting construct contained Section A (SEQ ID NO: 4) and the portion of Section B (SEQ ID NO: 5) up to the Neo^{R}insertion point (FIG. 2). The right arm of the targeting construct includes Section B (SEQ ID NO: 5) after the Neo insertion point and Section C (SEQ ID NO: 6) (FIG. 2). The resulting vector, pc155, was sequenced to ensure the vector was correctly assembled. The sequence of the site of Neo^{R}insertion is shown in FIG. 2. The Neo^{R}insertion deletes nucleotides 173 to 219 of murine CAR, which corresponds to amino acids 21-86 (GenBank Accession No. AF009327).

B. Creation of Embryonic Stem Cells Containing a Disrupted CAR Allele

Embryonic stem cells that contain a disrupted CAR allele were prepared as follows.

1. Electroporation

The pc155 vector was electroporated into E14 embryonic stem (ES) cells, which are derived from 129/Ola mice, as follows:

1. Gelatinize eight 100 mm plates.

2. Remove media from ES cell plate(s) and rinse once with 1× phosphate buffered saline (PBS).

3. Trypsinize cells with 3-5 ml 2× Trypsin/EDTA.

4. Pipette vigorously to get a single cell suspension.

5. Transfer all cells into 50 ml tube containing 13 ml ES media. Fill the tube with ES medium to 50 ml total, so as to obtain a 2:1 ratio of ES medium: 2× trypsin/EDTA.

6. For electroporation: Use 10×10⁶ cells in 400 μl.

7. Transfer the cells into a 2 mm gap cuvette.

8. Immediately after electroporation, put cells in 8 ml ES media. Add 20 μg linearized DNA.

9. Place cuvette on ice five minutes.

10. Electroplate on BTX electroporator. Parameters: 400 μF resistance; 200 volts.

11. Put 10 ml ES medium into each 100 mm dish.

12. Put 1 ml electroporated cells/100 mm dish.

13. 48 hours later, change the media to ES media +G418 (200 μg/ml).

14. Change the media to fresh ES media +G418 (200 μg/ml) 4, 6 and 8 days after electroporation.

The ES cells were selected for antibiotic resistance conferred by the selectable marker in the pc155 vector by growing the cells in G418 (200 μg/ml).

2. Identification of ES Cells that Contain the CAR Targeting Sequence

Hybridization analysis was used to screen the G418-resistant colonies to identify cells into which the CAR targeting sequences were integrated. The following overlapping oligos were used to create the hybridization probe:

| | | |
|---|---|---|
| Oligo 2930: | 5′CCATAAACGTGTTGATATCTGCAAAGTGTGCGAGCAGA | (SEQ ID NO: 10) |
| | GGCAACACGGGGCCCCGAGG. | |
| Oligo 2931 | 5′CTCTACAGCCTCCAGCCTATCTGTTCATGCATCACCGGCC | (SEQ ID NO: 11) |
| | TTTCCAGCCTCGGGGCCC3′. | |

The hybridization analysis was conducted using the following protocol:

1. Beginning with a 100 μM stock of each oligo, add 1 μl of each oligo to 35 μl of ultrapure water.

2. Add 1 μl of the diluted oligo pair to 7 μl ultrapure water.

3. Denature for 2 min and place on ice for 2 min.

4. Add 1 μl 0.5 mM dGTP, dTTP, 12.5 μl RH buffer, 5 μl each α-³²P-dATP and α-³²P-dCTP at 3000 Ci/mMol, and 1 μl (5 units) Klenow fragment and incubate 2 hr at room temp.

5. Purify through a G-50 Sephadex spin column, add to herring sperm DNA, denature, and add to blot with hybridization buffer. Hybridize overnight at 42° C.

6. Wash 3× for 15 min at 55° C. in 2× SSC 0.1% SDS.

7. Wash 3× for 5 min at 55° C. in 0.2 × SSC 0.1% SDS.

8. Expose for 3 days with BioMax® film (Kodak) and BioMax screens (Kodak).

| | Prehybridization and |
|---|---|
| RH Buffer | Hybridization Buffer |
| 0.5 M Hepes | 50% formamide |
| 12.5 mM MgCl₂ | 6X SSC |
| 30 mM 2-mercaptoethanol | 5X Denhardt’s solution |
| 0.125 M Tris-HCl pH 8 | 1% SDS |
| 1 mg/ml BSA | 10 mM NaPO₄ pH 7.4 |
| | 100 μg/ml herring sperm DNA |

3. Blastocyst Injection And Breeding Protocol

G418 cells that contained a proper disruption of a CARβ were used to generate chimeric mice in C57BL/6 host embryos. Blastocyst injection was performed according to standard procedures (see, e.g., Manipulating the Mouse Embryo, a Laboratory Manual, Hogan et al., Cold Spring Harbor Laboratory Press, Cold Spring Harbor, USA). Briefly, approximately 150 C57BL/6 blastocysts were injected with targeted CARP-disrupted ES cells, and reimplanted into CD-1 pseudopregnant female recipients. The number of pups was counted at the expected date of birth, and observed daily until weaning.

At weaning, the chimeras were assigned an individual identification number and earnotched following the numbering system found in Hogan et al., supra. The chimeras were scored, i.e., the proportion of ES cell contribution to the chimera was evaluated semi-quantitatively by estimating the percentage of non-black (agouti, chinchilla and albino) coat-color. The chimeras were generated with targeted E14 ES cells. The chimeras were mated to C57B1/6 females. Consequently the resulting progeny (i.e., the Fl mice) were 50% 129/Ola and 50% C57B1/6. F1 males and females both heterozygous for the targeted mutation were intercrossed to produce the knockout and control mice. The F2 generation (i.e., progeny of the F1 intercrosses), are hybrid mice (129/Ola:C57B1/6).

In general, the chimeras were not backcrossed to 129 substrains of mice, because 129 substrains are known to be heavily contaminated (i.e., not inbred). In general, it is recommended to breed chimeras to C57B1/6 for five generations or more and have the mutation in a C57B1/6 inbred background.

Hybridization analysis of tail DNA as described above was performed to genotype the chimeric mice and the offspring of the chimeric mice. Hybridization of the CAR probe to mRNA obtained from mice that are homozygous for the CARβ disruption revealed that no CARβ RNA was produced by these mice. The phenotypes described were found linked to mice bearing disruptions in both CAR alleles.

### Example 2

### Assay of Cholesterol Levels in Mice with CAR Gene Disruptions

Serum samples were withdrawn from 16 week-old age-matched mice (prepared as described in Example 1) that were wild-type, heterozygous for CAR allele disruptions, and homozygous for CAR allele disruptions. The level of serum cholesterol was assayed. The results are displayed in Table 2.

TABLE 2

| | | | Cholesterol |
|---|---|---|---|
| # | Genotype | Sex | mg/dl |
| 1 | Wild-type | Male | 71 |
| 2 | Wild-type | Female | 57 |
| 3 | Heterozygous | Male | 113 |
| 4 | Heterozygous | Female | 68 |
| 5 | Homozygous | Male | 99 |
| 6 | Homozygous | Male | 141 |
| 7 | Homozygous | Male | 134 |
| 8 | Homozygous | Female | 102 |
| 9 | Homozygous | Female | 112 |
| 10 | Homozygous | Female | 125 |

The level of serum cholesterol was not elevated in the female that was heterozygous for the disrupted CAR allele. The level of serum cholesterol, however, was elevated in the homozygous male. The level of serum cholesterol in all mice homozygous for both CAR allele disruptions was elevated over that of wild-type mice.

This experiment was repeated on larger sample number; these results indicated that the level of cholesterol is elevated approximately 20% in mice that are homozygous for the disrupted CAR allele as compared to wild-type mice. The results obtained using the larger sample number also indicated that the elevation of serum cholesterol was only seen in mice that have disruptions in both CAR alleles.

### Example 3

### Identification of a Naturally Occurring CAR Ligand: 5β-pregnan-3,20 dione

In this Example, a peptide sensor assay was used to identify a naturally occurring ligand for CARα, the human CAR.

A portion of DNA encoding the ligand binding domain of CARα encoding amino acids 76 to 348 (GenBank Accession No. CAA83016) was subcloned into a glutathione S-transferase (GST) fusion protein expression vector (pGEX-2TK, Amersham-Pharmacia) to create a vector that encodes a fusion protein in which GST is fused to the amino terminus of the CAR ligand binding domain. The GST-CAR protein was expressed in E. coli and purified over a glutathione affinity column. The GST-CAR fusion protein was incubated with a rhodamine-labeled peptide derived from the receptor binding domain of the coactivator SRC-1, rhodamine-ILRKLLQE (SEQ ID NO: 7) (see WO 99/27365). The fluorescence polarization was measured as described in WO 99/27365.

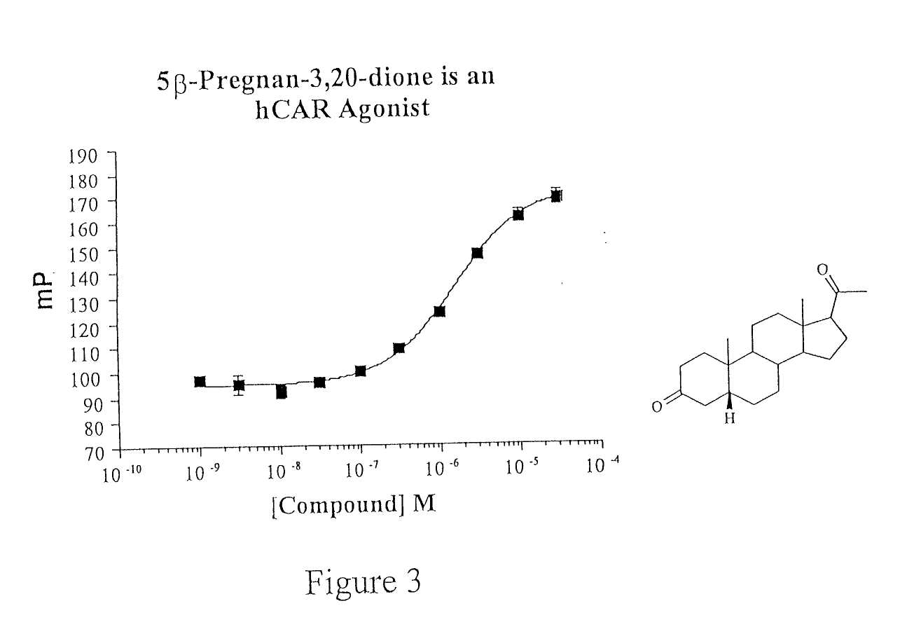

When 5β-pregnan-3,20-dione was included in the incubation mixture, the amount of fluorescence polarization between the sensor peptide and the GST-CAR increased (FIG. 3). The binding was saturable. This indicates that 5β-pregnan-3,20-dione is an agonist, as it promotes the association of the coactivator-derived sensor peptide to GST-CAR.

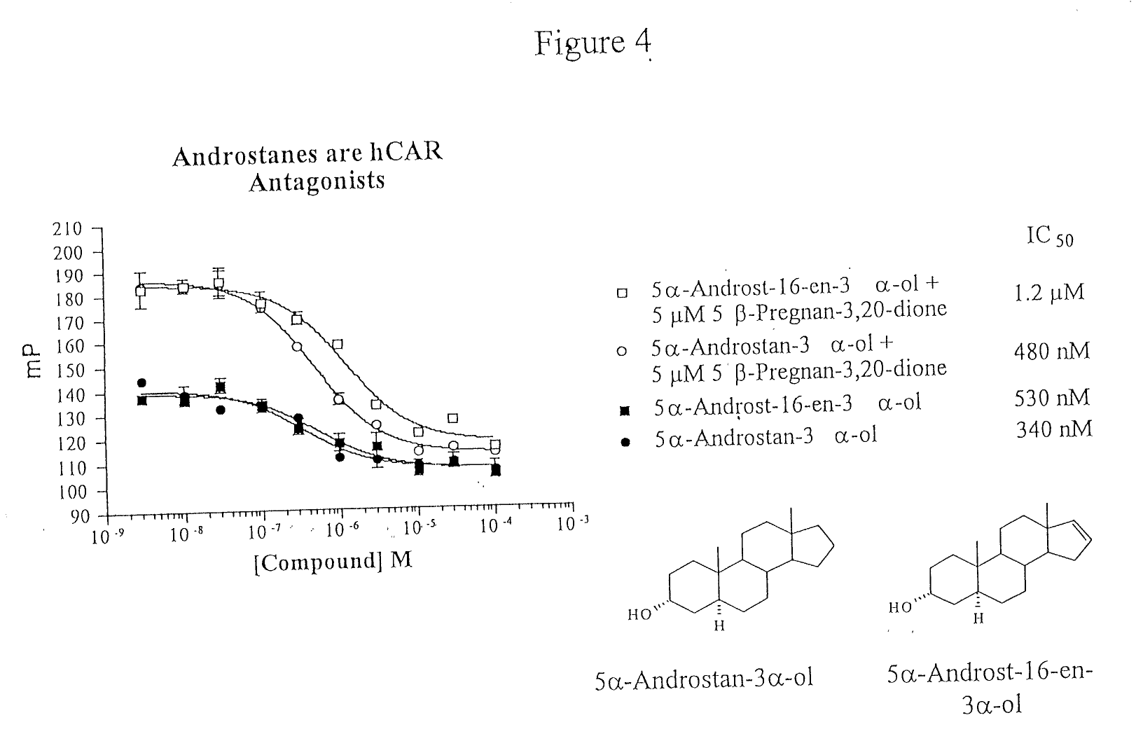

The androstanes, on the other hand, were found to be antagonists. When the androstanes 5α-androst-16-en-3α-ol and 5α-androstane-3α-ol were incubated with the sensor peptide and the GST-CAR fusion protein, the amount of fluorescence polarization (FP) decreased. The IC₅₀'s were 1.2 mM for 5α-androst-16-en-3α-ol and 480 nM for 5α-androstane-3α-ol. The androstane compounds were also able to decrease the FP seen when 5 μM 5β-pregnan-3,20-dione was also present in the incubation mixture along with the sensor peptide and the GST-CAR fusion protein (FIG. 4). The IC₅₀s for 5α-androst-16-en-3α-ol and 5α-androstane-3α-ol in this experiment were 530 nM and 340 nM, respectively (FIG. 4). These results indicate that these androstanes are CAR antagonists in that they reduce the interaction of CAR with the SRC-1 coactivator-derived sensor peptide.

### Example 4

### 5β-pregnan-3,20 Dione Increases CAR Transcription

This Example demonstrates that 5β-pregnan-3,20-dione, which was shown to be a CAR agonist in the peptide sensor assay of Example 3, stimulates transcription of a reporter gene that is linked to a CAR-mediated retinoic acid response element (RARE). The reporter plasmid had the RARE operably linked to a thymidine kinase promoter, which in turn drives expression of a luciferase reporter gene (pRAREβ-tk luc).

CV-1 cells were plated in DME medium supplemented with 10% charcoal stripped calf serum at a density of 120,000 cells per well in a 24 well plate (Costar) 16-24 h before transfection. Approximately 100 ng of pRAREβ-tk luc, 50 ng of a control β-galactosidase expression vector (pCMVβ, Clontech), and increasing amounts of pCMV-CARβ mammalian expression vector were mixed with carrier DNA (pBluescript, Stratagene) to a total of 500 ng of DNA per well in a volume of 50 μl OptiME medium (GIBCO BRL). To this mixture was added a second mix containing 45 μl of OptiME medium and 5 μl of LipoFectamine (GIBCO BRL). After a 30 min. incubation period, an additional 400 μl of OptiME medium was added and the combined mix was then applied to the cells. Sixteen hours later the medium was exchanged to DME medium supplemented with 10% dilapidated fetal calf serum (Sigma) and the test compound (vehicle, 5α-androstane-3α-ol, or 5β-pregnan-3,20-dione) at a concentration of 10 μM. After incubation for an additional 24 h, luciferase activity and β-galactosidase activity were measured using standard procedures. The luciferase activity was reported as normalized luciferase activity which is the luciferase activity in relative light units (RLU) divided by the amount of β-galactosidase activity.

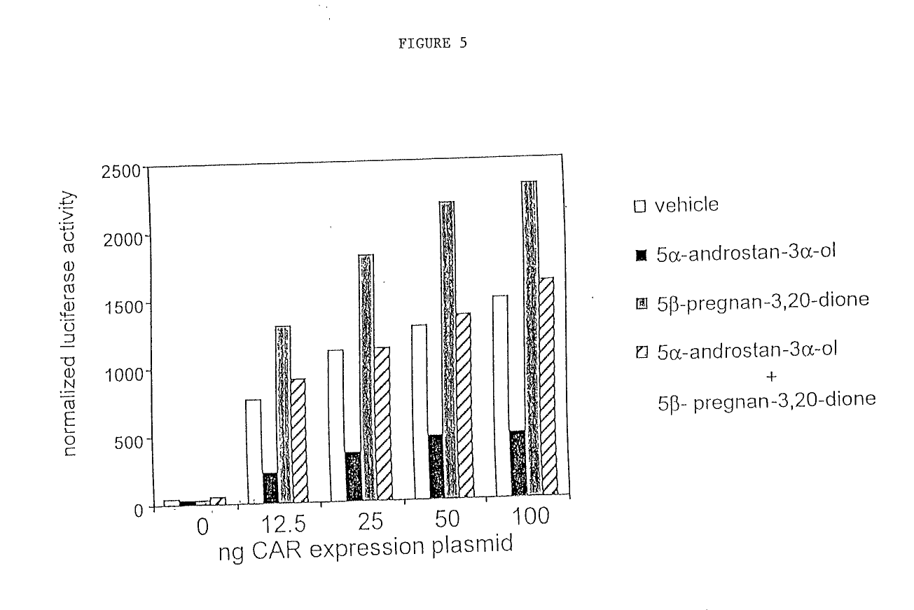

Increasing amounts of CAR expression plasmid resulted in an increase in the normalized luciferase activity (FIG. 5). This demonstrates that CAR is able to activate transcription from the retinoic acid response element. The CAR agonist 5β-pregnan-3,20-dione resulted in increased transcription of the luciferase reporter gene when included in the medium. In contrast, when the 5α-androstane-3α-ol was included in the medium, the amount of transcription decreased (FIG. 5). This is consistent with the antagonist role for the androstanes in the peptide sensor assays described above. Interestingly, the amount of basal CAR transcription could be increased with the addition of 10 μM 5β-pregnan-3,20-dione.

### Example 5

### Tolerance of Mice to Fat/Lipid Challenge

Mice were created as in Example 1. Age-matched mice homozygous for the CAR disruption and homozygous for the wild-type CAR allele were fed Picolab 5053 (containing 4% fat) or a high fat diet (Picolab 5053 containing 1.25% cholesterol and 10% coconut oil). Blood was withdrawn from the mice after 0, 7, 15, and 21 days on the diet. Total cholesterol was determined by fractionating the serum on FPLC using a Sepharose 4B column for particle size exclusion. The cholesterol content of the fractions was determined using a Hitachi clinical analyzer.

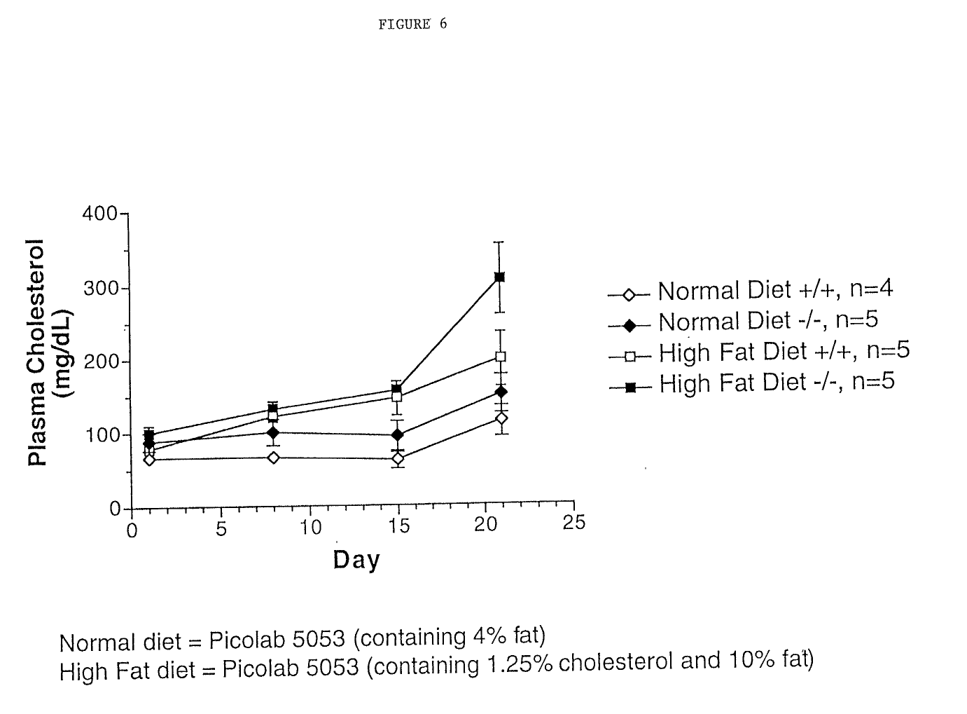

The mice on the normal diet showed no difference in their serum cholesterol levels (FIG. 6). However, mice that were homozygous for CAR allele disruption showed a increase in serum cholesterol over wild-type mice at 21 days. These results indicate that mice homozygous for the CAR allele disruption exhibit a reduced tolerance to maintain plasma cholesterol levels when challenged with a high-fat, high cholesterol diet.

### Example 6

### The Effects of TCPOBOP on Lipid Levels in CAR Deficient Mice

A xenobiotic compound, TCPOBOP (1,4-bis[2-(3,5-dichloropyridyloxy)]benzene), is a potent mouse CAR ligand and activates CAR in transcription assays and upregulates CAR-mediated transcription with an EC₅₀ value of 20 nM (Tzameli et al. (2000) Mol. Cell. Biol. 20: 2951-2958). Further, TCPOBOP induces half maximal expression of the CAR target gene CYP2B (Honkakoski et al., (1998) Mol. Cell. Biol. 18: 5652-5658) in mice at a dose of 66 μg/kg applied by intraperitoneal injection. A single intraperitoneal injection of TCPOBOP at 3 mg/kg induces CYP2B expression for over 20 weeks (Poland et al., (1980) Mol. Pharmacol. 18: 571-580). Because TCPOBOP is highly selective, binds murine CAR with good potency, and has an excellent pharmacokinetic profile, it was examined for its ability to decrease cholesterol levels, in wild-type, but not CAR deficient mice.

Mice were created as in Example 1. CAR knockout mice, CAR mice heterozygous for a CAR gene disruption, and wild-type mice received a single 0.2 ml intraperitoneal injection of TCPOBOP at a concentration of 0.3 mg/ml in corn oil, at a dose of 3 mg/kg or a 0.2 ml intraperitoneal injection of vehicle (corn oil) alone. Each treatment group contained two animals. Four days after dosing, the mice were sacrificed by carbon dioxide inhalation.

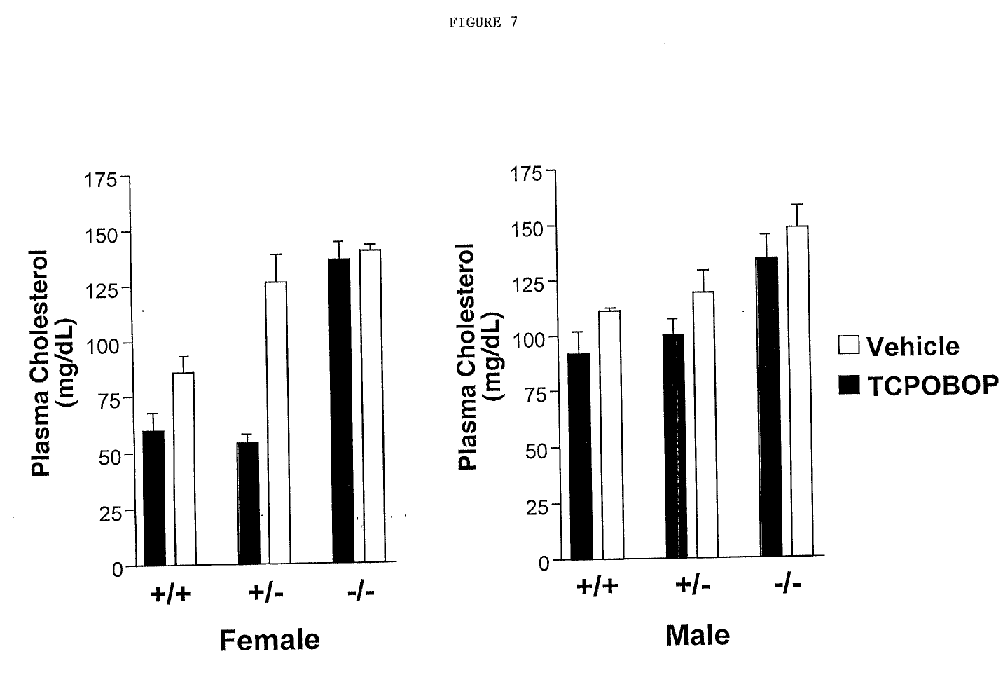

Using a Roche clinical analyzer, plasma samples were analyzed for total plasma cholesterol. The TCPOBOP treatment decreased total plasma cholesterol, in wild-type littermates (+/+) (FIG. 7) and in mice that contain only one copy of a functional CAR gene (+/−) (FIG. 7). The most pronounced effect was seen in female heterozygous CAR mice (+/−), which showed a 60% reduction in total cholesterol in response to the TCPOBOP treatment (FIG. 7). However, no effect on plasma cholesterol levels with TCPOBOP treatment was observed in CAR knockout mice (FIG. 7).

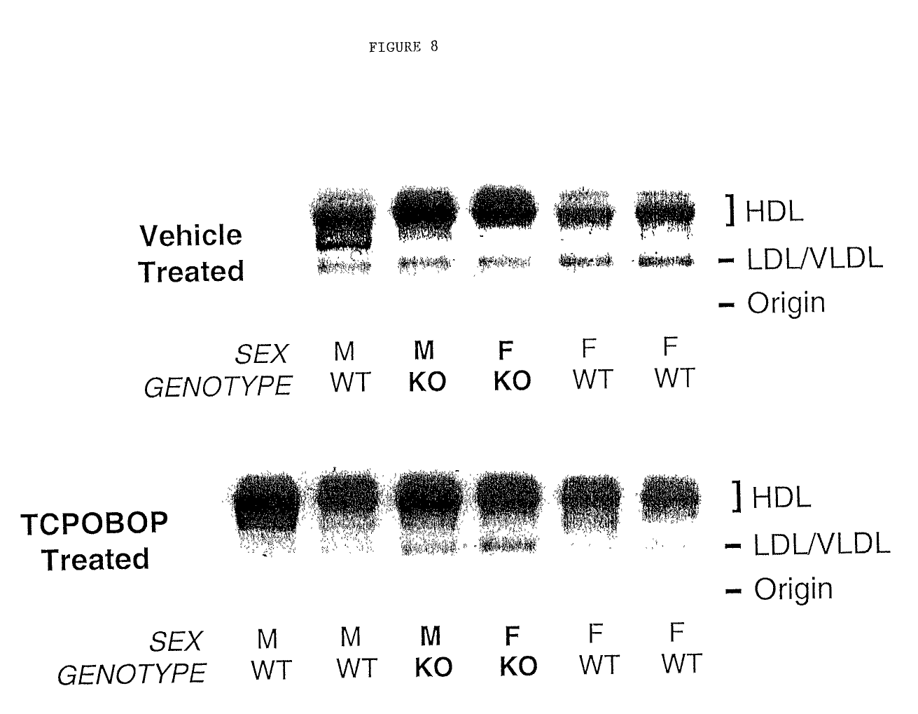

Next, the effects of TCPOBOP on the levels of LDL/VLDL or HDL particles was assayed. For determination of lipoprotein particle composition, plasma samples were separated by electrophoresis using the Paragon® Lipoprotein Electrophoresis Kit (Beckmann Instruments, Inc., Fullerton, Calif.) and stained according to the manufacturer's instructions. Consistent with the increase in total plasma cholesterol seen in this Example in CAR knockout mice and in Example 2, the determination of lipoprotein particle composition showed that CAR knockout mice exhibit increased amounts of HDL particles as compared to wild-type mice (see, vehicle treated panel in FIG. 8). No significant changes were observed between the amounts of LDL/VLDL in CAR knockout mice as compared to their wild-type littermates (see, vehicle treated panel in FIG. 8).

Interestingly, TCPOBOP treatment caused a significant reduction in plasma levels of LDL/VLDL in wild-type mice but not in knockout mice (see, TCPOBOP treated panel in FIG. 8). The bands corresponding to LDL/VLDL are not present in the TCPOBOP treated wild-type mice, but are unaffected in the CAR deficient mice as compared to the vehicle control. These results indicate that TCPOBOP is acting through a CAR-mediated process to decrease cholesterol levels. These results also indicate that a CAR activator has the ability to decrease total plasma cholesterol by mainly lowering the “bad cholesterol” LDL/VLDL. Reducing plasma levels of LDL is an important factor in reducing the risk of clinical events associated with human coronary artery disease (Witzum (1996) Chapter 36, Drugs Used in the Treatment of Hyperlipoproteinemias, in Hardman et al., eds. Goodman & Gilman 's The Pharmacological Basis of Therapeutics 9th ed.). Thus, compounds that act through a CAR-mediated process are useful as pharmaceutical agents.

### Example 7

### The Effects of TCPOBOP on Lipid Levels in Wild-type C57B1/6 Mice

It is known that the mouse strain C57B1/6 expresses elevated levels of plasma lipids. In addition, mice are known to carry most of their cholesterol in the form of HDL particles and most of the triglyceride in the plasma is contained in form of VLDL particles. Therefore, the effects of TCPOBOP on plasma cholesterol, HDL cholesterol and triglycerides levels in wild-type C57B1/6 mice were also analyzed. Wild-type C57B1/6 mice received a single intraperitoneal injection of TCPOBOP in corn oil at a dose of 3 mg/kg or vehicle (corn oil) alone. Four days after dosing, the mice were sacrificed by carbon dioxide inhalation. Using a Roche clinical analyzer, plasma samples were analyzed for total plasma cholesterol, HDL cholesterol, and triglyceride levels with the Boehringer Mannheim reagents Cholesterol/HP, HDL-C plus, and Triglycerides/GPO, respectively.

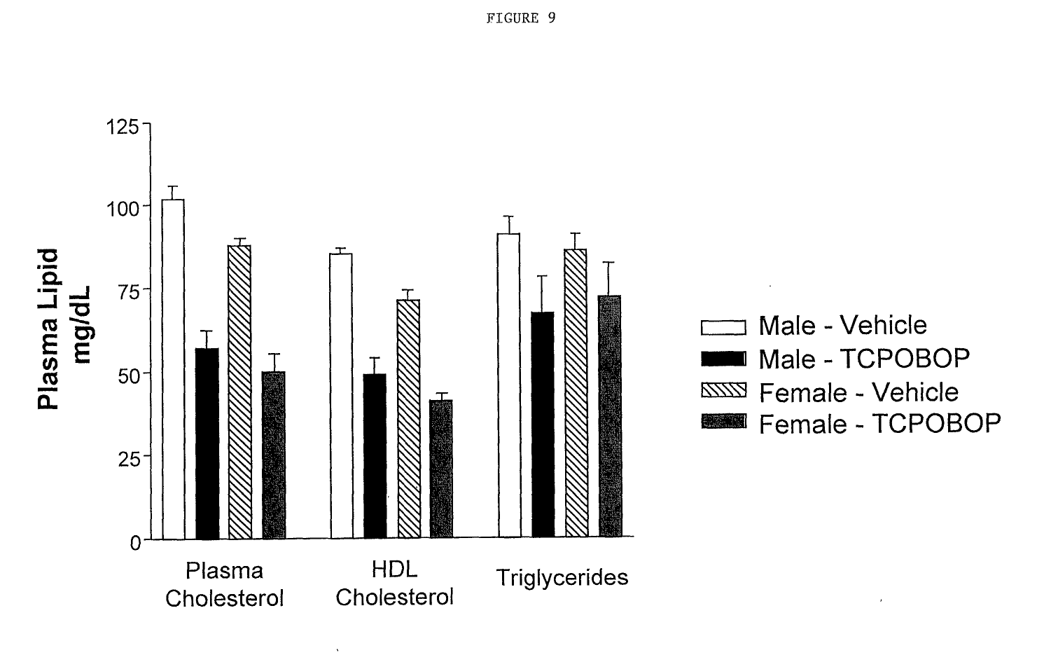

TCPOBOP treatment resulted in a 40% reduction in total plasma cholesterol and a 40% reduction in HDL (FIG. 9). Consistent with the observation that TCPOBOP lowers LDL/VLDL levels in FIG. 8, TCPOBOP treatment of C57B1/6 mice also led to a 15-30% reduction in triglyceride levels (FIG. 9).

### Example 8

### Analysis of Candidate Therapeutic Agents in the Peptide Sensor Assay

Using the compounds set out in Table 1, a peptide sensor assay was carried out using the method of Example 3. Fusion proteins encoding the ligand binding domain of CARα (GST-CARα; see Example 3) and CARβ (GST-CARβ) were used in the assay. A portion of the DNA for the ligand binding domain of CARβ (GenBank Accession No. AF009327) encoding amino acids 86 to 358 was subcloned into a glutathione S-transferase (GST) fusion protein expression vector (pGEX-2TK, Amersham-Pharmacia) to create a vector that encodes a fusion protein in which GST is fused to the amino terminus of the CARβ ligand binding domain (GST-CARβ). The GST-CARβ was purified using glutathione affinity chromatography.

The molar EC₅₀'s are listed in Table 3. The notation ND means not determined. The EC₅₀ is the half-maximal concentration at which full stimulation of the peptide sensor assay signal is exhibited for that particular compound being tested as a candidate therapeutic agent.

TABLE 3

| Compound | GST-CARα EC₅₀ (M) | GST-CARβ EC₅₀ (M) |
|---|---|---|
| TCPOBOP | ND | 6.00 × 10⁻⁸ |
| 5β-pregnan-3,20-dione | 2.50 × 10⁻⁶ | ND |
| T0438248 | 5.25 × 10⁻⁹ | 6.54 × 10⁻⁸ |
| T0434523 | 7.97 × 10⁻⁸ | 5.22 × 10⁻⁸ |
| T0318784 | 9.12 × 10⁻⁸ | 9.67 × 10⁻⁸ |
| T0037566 | 2.21 × 10⁻⁷ | 9.91 × 10⁻⁸ |
| T0509562 | 2.57 × 10⁻⁷ | 1.55 × 10⁻⁸ |

The EC₅₀'s for the compounds listed in Table 1 are all lower than that of the natural ligand 5β-pregnan-3,20-dione (Table 3). These results indicate that TCPOBOP, T0438248, T0434523, T0318784, T0037566, and T0509562 can act as CAR agonists as they promote the association of the coactivator-derived sensor peptide to GST-CARα and GST-CARβ.

### Example 9

### Analysis of Candidate Therapeutic Agents in a Transcription Assay

The compounds of Table 1 were examined for their ability to stimulate CAR-mediated expression of a RARE linked to a luciferase reporter plasmid as in Example 4. The plasmids pCMV-CARα (50 ng) and pCMV-CARβ (50 ng) were used in the reporter gene assay of Example 4 in the presence of 10 μM. of the compounds of Table 1 (T0438248, T0434523, T0318784, T0037566 and T0509562). The luciferase activity is reported as the fold increase in luciferase activity over a DMSO (vehicle) control. Luciferase acivity is normalized by dividing luciferase activity measured in relative light units (RLU) by the amount of β-galactosidase activity.

TABLE 4

| Compound | CMV-CARα | CMV-CARβ |
|---|---|---|
| T0438248 | 2.92 | 0.96 |
| T0434523 | 2.17 | 1.29 |
| T0318784 | 1.78 | 3.87 |
| T0037566 | 1.96 | 2.14 |
| T0509562 | 1.07 | 1.94 |

These results demonstrate that the compounds of Table 1 are able to activate transcription of a RARE reporter gene through CARα or CARβ.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for identifying a therapeutic agent for use in treating a CAR-mediated disorder or condition, the method comprising:
identifying a candidate therapeutic agent by screening one or more compounds to determine whether said compounds can modulate a CAR-mediated intermolecular interaction;
administering the candidate therapeutic agent to a test mammal; and
determining whether the level of a cholesterol indicator is modulated in said test mammal.

2. The method of claim 1, wherein said candidate therapeutic agent is 5β-pregnan-3,20-dione.

3. The method of claim 1, wherein said CAR-mediated disorder or condition is selected from the group consisting of: hypercholesterolemia, lipid disorders, atherosclerosis, and cardiovascular disorders.

4. The method of claim 1, wherein the mammal is a cholesterol-elevated mammal.

5. The method of claim 4, wherein the test mammal has a disruption in both CAR alleles.

6. The method of claim 1, wherein said cholesterol indicator is the level of serum cholesterol.

7. The method of claim 1, wherein said cholesterol indicator is the level of a member selected from the group consisting of HDL cholesterol, LDL cholesterol, and VLDL cholesterol.

8. The method of claim 1, wherein said cholesterol indicator is the mRNA level of a gene involved in the regulation of cholesterol levels.

9. The method of claim 1, wherein said CAR-mediated intermolecular interaction is CAR-mediated gene expression.

10. The method of claim 9, wherein the ability of said candidate therapeutic agent to modulate CAR-mediated gene expression is assessed by:
providing a cell that comprises:
a) a polynucleotide encoding a fusion polypeptide that comprises: 1) an amino acid sequence that comprises a DNA binding domain of a polypeptide; and 2) a ligand binding domain that is substantially identical to a ligand binding domain of CAR; and
b) a reporter gene construct which comprises a response element to which the DNA binding domain can bind, wherein the response element is operably linked to a promoter that is operative in the cell and the promoter is operably linked to a reporter gene; and
contacting said cell with said candidate therapeutic agent; and
determining whether said reporter gene is expressed at a higher or lower level in the presence of said candidate therapeutic agent as compared to expression in the absence of said candidate therapeutic agent.

11. The method of claim 10, wherein said candidate therapeutic agent is 5β-pregnan-3,20-dione.

12. The method of claim 10, wherein said DNA binding domain is substantially identical to a DNA binding domain from a polypeptide selected from the group consisting of: CAR, a GAL4 transcription factor, an estrogen receptor, a progesterone receptor, a glucocorticoid receptor, an androgen receptor, a mineralcorticoid receptor, a vitamin D receptor, a retinoid receptor, and a thyroid hormone receptor.

13. The method of claim 12, wherein said DNA binding domain is a CAR DNA binding domain and the response element is a CAR response element.

14. The method of claim 13, wherein said CAR response element is a DR-5 element or a DR-4 element.

15. The method of claim 10, wherein said reporter gene encodes a marker protein selected from the group consisting of: luciferase, alkaline phosphatase, beta-galactosidase, chloramphenicol acetyltransferase and green fluorescent protein.

16. The method of claim 1, wherein said CAR-mediated intermolecular interaction is the binding of a polypeptide that comprises a CAR ligand binding domain to a ligand for CAR.

17. The method of claim 16, wherein said polypeptide is a CARα or a CARβ.

18. The method of claim 16, wherein said ligand for CAR comprises a sensor peptide.

19. The method of claim 18, wherein said ligand for CAR comprises a receptor binding domain of a coactivator or a corepressor.

20. The method of claim 19, wherein said coactivator is SRC-1.

21. The method of claim 20, wherein said sensor peptide is rhodamine labeled ILRKLLQE.

22. The method of claim 16, wherein the binding of the polypeptide that comprises a CAR ligand binding domain to the ligand for CAR is determined in the presence of a naturally occurring ligand for CAR.

23. The method of claim 22, wherein said naturally occurring ligand for CAR is 5β-pregnan-3,20-dione.

24. The method of claim 16, wherein said method comprises determining whether said compound can inhibit the interaction between the CAR ligand binding domain and the CAR ligand.

25. The method of claim 24, wherein said CAR ligand is labeled.

26. The method of claim 25, wherein said CAR ligand is radiolabeled.

27. The method of claim 24, wherein said CAR ligand is labeled with a fluorophore.

28. The method of claim 27, wherein said method comprises a fluorescence polarization assay.

29. The method of claim 27, wherein said method comprises a fluorescence resonance energy transfer assay.

30. The method of claim 27, wherein said CAR is labeled with a fluorophore.

31. The method of claim 30, wherein said method comprises a fluorescence resonance energy transfer assay or a fluorescence polarization assay.

32. The method of claim 24, wherein said CAR ligand is selected from the group consisting of:
5α-androst-16-en-3α-ol, 5α-androst-16-en-3α-ol acetate, 5α-androstane-3α-ol, 5α-androst-16-en-3α-ol acetate and 5β-pregnan-3,20-dione.

33. A method for identifying a therapeutic agent for use in treating a CAR-mediated disorder or condition the method comprising:
administering a compound to a CAR compromised mammal; and
determining whether administration of the compound results in a change in cholesterol level compared to a mammal to which the compound is not administered.

34. The method of claim 33, wherein the method further comprises administering the compound to a CAR non-compromised mammal and comparing the effect on the cholesterol level indicator of administering the compound to that of administering the compound to the CAR compromised mammal.

35. The method of claim 33, wherein said cholesterol level indicator is the level of serum cholesterol.

36. The method of claim 33, wherein said cholesterol level indicator is the level of a member selected from the group consisting of HDL cholesterol, LDL cholesterol, and VLDL cholesterol.

37. The method of claim 33, wherein said cholesterol level indicator is the mRNA level of a gene involved in the regulation of cholesterol levels.

38. The method of claim 33, wherein said CAR compromised mammal is a mammal having a disruption in both CAR alleles.

39. The method of claim 38, wherein said CAR compromised mammal is a mouse.

40. The method of claim 38, wherein said disruption occurs in the coding region for the DNA binding domain of CAR.

41. The method of claim 38, wherein said disruption in a CAR allele comprises an insertion at codons for amino acid positions from about amino acid 21 to about amino acid 86 of CARβ.

42. A method for treating a CAR-mediated disorder or condition, the method comprising:
administering to a mammal having a CAR-mediated disorder or condition an effective amount of a therapeutic agent that modulates CAR-mediated regulation of cholesterol levels.

43. The method of claim 42, wherein said therapeutic agent is identified by:
screening one or more compounds to determine whether said compounds can modulate a CAR-mediated intermolecular interaction;
administering the candidate therapeutic agent to a test mammal; and
determining whether the level of a cholesterol indicator is affected in said test mammal.

44. The method of claim 42, wherein said CAR-mediated disorder or condition is selected from the group consisting of: hypercholesterolemia, lipid disorders, atherosclerosis, and cardiovascular disorders.

45. A non-human mammal having a genome that comprises a disruption in at least one CAR allele.

46. The non-human mammal of claim 45, wherein said disruption comprises an insertion, deletion or mutation in a region of the CAR allele that encodes for a DNA binding domain of CAR.

47. The non-human mammal of claim 46, wherein said disruption comprises an insertion at codons for amino acid positions from 21 to about 86 of CARβ.

48. A non-human mammal having a genome that comprises a disruption in both CAR alleles.

49. The non-human mammal of claim 48, wherein said disruption comprises an insertion, deletion or mutation in a region of the CAR allele that encodes for a DNA binding domain of CAR.

50. The non-human mammal of claim 48, wherein said disruption comprises an insertion at codons for amino acid positions from 21 to about 86 of CARβ.

51. The non-human mammal of claim 48, wherein said non-human mammal exhibits an increased level of serum cholesterol relative to a wild-type mammal.

52. A method for producing a transgenic non-human mammal having a genome that comprises a disrupted CAR allele, the method comprising:
introducing into embryonic stem cells a polynucleotide that comprises a coding region for a portion of a CAR polypeptide, wherein the polynucleotide sequence includes a disruption in the coding region of a portion of said CAR polypeptide;
identifying a cell into which said polynucleotide sequence has been integrated into an endogenous CAR allele;
introducing said cell into a blastocyst, thereby forming a transgenic blastocyst;
implanting said transgenic blastocyst into a pseudopregnant mammal and allowing said pseudopregnant mammal give birth to a transgenic mammal.

53. The method of claim 52, wherein said transgenic mammal contains a disrupted CAR allele in its germline.

54. The method of claim 53, further comprising breeding said transgenic mammal to generate a heterozygous mammal comprising a disrupted CAR allele.

55. The method of claim 53, further comprising mating a male and a female mammal each heterozygous for said disrupted CAR allele, to form progeny that are homozygous for said disrupted CAR allele.

56. The method of claim 52, wherein said disrupted CAR allele comprises an insertion into a region of the CAR allele that codes for a DNA binding domain of CAR.

57. The method of claim 52, wherein said disrupted CAR allele comprises an insertion at codons for amino acid positions from about 21 to about 86 of CARβ.

58. The method of claim 56, wherein said insertion comprises a selectable marker gene.

59. The method of claim 58, wherein said marker gene encodes for neomycin resistance.
